(19) United States
(12) Patent Application Publication
(10) Pub. No.: US 2013/0177577 A1
(43) Pub. Date: Jul. 11, 2013

(54) NLRC5 AS A TARGET TO INTERVENE MHC CLASS 1-MEDIATED IMMUNE RESPONSES

(71) Applicants: Koichi S. Kobayashi, Chestnut Hill, MA (US); Torsten B. Meissner, Boston, MA (US); Amy Li, Boston, MA (US)

(72) Inventors: Koichi S. Kobayashi, Chestnut Hill, MA (US); Torsten B. Meissner, Boston, MA (US); Amy Li, Boston, MA (US)

(73) Assignee: Dana-Farber Cancer Institute, Inc., Boston, MA (US)

(21) Appl. No.: 13/808,729

(22) PCT Filed: Jul. 12, 2011
(86) PCT No.: PCT/US11/43681

§ 371 Date: Mar. 27, 2013

Related U.S. Application Data
(60) Provisional application No. 61/363,393, filed on Jul. 12, 2010.

(51) Int. Cl. A61K 48/00; A61K 45/06; A61K 36/00; A61K 35/12; A61K 38/02; A61K 35/66

(52) U.S. Cl. CPC A61K 48/00; A61K 38/02; A61K 35/66; A61K 36/00; A61K 35/12; A61K 45/06
USPC 424/172.1; 514/44 A; 514/1.1; 424/780; 424/725; 424/520; 506/7; 435/6.12; 435/7.21

(57) ABSTRACT

A method to modulate MHC class I gene expression by modulating NLRC5 expression and/or NLRC5 activity in a subject is provided. The method comprises administering to the subject a compound HLA-A HLA-B that modulates NLRC5 expression and/or NLRC5 activity in an amount effective to modulates MHC class I gene expression. Also described is a screen for compounds that modulate NLRC5 expression. Candidate compounds are tested for their ability to modulate NLRC5 expression.

### RELATED APPLICATIONS

This application claims the benefit under 35 U.S.C. §119(e) of U.S. provisional application Ser. No. 61/363,393, filed Jul. 12, 2010, the content of which is incorporated by reference herein in its entirety.

### FEDERALLY SPONSORED RESEARCH

This invention was made with government support under R01DK074738 awarded by National Institute of Health. The government has certain rights in the invention.

### BACKGROUND OF THE INVENTION

Major histocompatibility complex (MHC) class I and class II molecules play essential roles in the activation of adaptive immune responses by presenting antigens to T lymphocytes. The ability of T lymphocytes to recognize and kill infected cells is mediated by MHC complexes that display fragmented pieces of self or non-self antigens on the host's cell surface. There are two general class of MHC molecules: MHC class I molecules, which are found on almost all nucleated cells, and MHC class II molecules, which are found on certain immune cells. MHC class II molecules present foreign particles degraded by phagocytic cells such as macrophages, neutrophils and monocytes. The presentation of MHC class I complexes and their recognition by CD8+ T lymphocytes has been implicated in a variety of human and animal conditions, including infectious diseases, cancer, autoimmunity and transplantation rejections. MHC Class I complexes appear to be of particular importance in skin graft rejection (Zijlstra, M., Auchincloss, H., Loring, J., Chase, C., Russell, P., and Jaenisch, R., J. Exp. Med. 175:885-893 (1992)). In addition, a large number of autoimmune diseases are believed to be the result of CD8+ T lymphocytes attacking cells displaying MHC class I complexes. For example, there is evidence that attack by CD8+ T lymphocytes plays a role in multiple sclerosis (Steinman, L., Autoimmune disease Sci. Amer. 269(3): 106-114), diabetes (Oldstone, M. B., A., Nerenberg, M., Southern, P., Price, J., and Lewicki, H., Cell 65:319-331 (1991)), and arthritis (Braun, W. E., Clin. Biochem. 25(3):187-191 (1992). It would consequently be desirable to be able to modulate the expression of MHC class I genes in order to treat or prevent diseases associated with an aberrant expression of MHC class I genes.

### SUMMARY OF THE INVENTION

As described herein, NLRC5 is a transcriptional regulator that orchestrates the concerted expression of critical components in the MHC class I pathway. Described herein is a method of modulating MHC class I gene expression by modulating NLRC5 expression in a subject. In one embodiment of the method, a compound that modulates NLRC5 expression and/or NLRC5 activity is used to modulate MHC class I gene expression. For example, a compound that modulates (increases or inhibits/reduces) NLRC5 expression and/or NLRC5 activity is administered to an individual in an amount sufficient to modulate (increase or inhibit/reduce) MHC class I gene expression. In specific embodiments, the method is carried out to reduce (partially or totally) viral infection in subjects who have been exposed to or are at a risk of being exposed to viral infections. In other embodiments, the method is carried out to treat cancer in individuals who have cancer or to reduce tissue or organ rejection in individuals in need thereof.

In one embodiment, the method is a method of modulating MHC class I gene expression by modulating NLRC5 expression and/or NLRC5 activity in a subject. The method comprises administering to the subject a compound that modulates NLRC5 expression and/or NLRC5 activity in an amount effective (sufficient) to modulate MHC class I gene expression. In some embodiments, the compound increases NLRC5 expression and/or NLRC5 activity, whereby MHC class I gene expression is increased. In other embodiments, the compound decreases NLRC5 expression and/or NLRC5 activity, whereby MHC class I gene expression is decreased. Examples of compounds that may be used include, but are not limited to, siRNA, a synthetic organic chemical, a peptide, a natural fermentation product, and a substance extracted from a microorganism, plant, animal or cell culture medium. In some embodiments, the method further comprises administering to the subject a compound that increases CIITA expression and/or CIITA activity in an amount effective to increase MHC class I and MHC class II gene expression. In some embodiments, the method further comprises administering to the subject a compound that decreases CIITA expression and/or CIITA activity in an amount effective to decrease MHC class I and MHC class II gene expression.

In one embodiment, the method is a method of reducing viral infection by increasing NLRC5 expression and/or NLRC5 activity in a subject in need thereof. The method comprises administering to the subject a compound that increases NLRC5 expression and/or NLRC5 activity in an amount effective to increase MHC class I gene expression and reduce the viral infection in the subject. Examples of compounds that may be used include, but are not limited to, siRNA, a synthetic organic chemical, a peptide, a natural fermentation product, and a substance extracted from a microorganism, plant, animal or cell culture medium. In some embodiments, the method further comprises administering to the subject a compound that increases CIITA expression and/or CIITA activity in an amount effective to increase MHC class I gene expression and reduce the viral infection in the subject.

A further embodiment is a method of inhibiting cancer by increasing NLRC5 expression and/or NLRC5 activity in a subject. The method comprises administering to the subject a compound that increases NLRC5 expression and/or NLRC5 activity in an amount effective to increase MHC class I gene expression and inhibit cancer in the subject. Examples of compounds that may be used include, but are not limited to, siRNA, a synthetic organic chemical, a peptide, a natural fermentation product, and a substance extracted from a microorganism, plant, animal or cell culture medium. In some embodiments, the method further comprises administering to the subject a compound that increases CIITA expression in an amount effective to increase MHC class I and MHC class II gene expression and inhibit cancer in the subject.

An additional embodiment is a method of inhibiting tissue or organ rejection by decreasing NLRC5 expression in a subject. The method comprises administering to the subject a compound that decreases NLRC5 expression and/or NLRC5 activity in an amount effective to decrease MHC class I gene expression and thereby inhibit tissue or organ rejection in the subject. Examples of compounds that may be used include, but are not limited to, siRNA, a synthetic organic chemical, a peptide, a natural fermentation product, and a substance extracted from a microorganism, plant, animal or cell culture medium. In some embodiments, the method further comprises administering to the subject a compound that decreases CIITA expression in an amount effective to decrease MHC class I and class II gene expression and inhibit tissue or organ rejection in the subject.

Screening methods to identify compounds that modulate NLRC5 expression and/or NLRC5 activity are also provided. Some embodiments are a method of identifying a compound that increases NLRC5 expression and/or NLRC5 activity. The method comprises (a) contacting a test cell with a test compound, wherein the cell comprises a NLRC5 nucleic acid; and (b) comparing the level of expression and/or activity of NLRC5 in the test cell to the level of expression and/or activity of NLRC5 in a cell, referred to as a control cell, that is the same type of cell, but has not been contacted with the test compound, wherein if the level of expression and/or activity of NLRC5 in the test cell is greater than the level of expression and/or activity in the control cell, the test compound is a compound that increases NlRC5 expression and/or NLRC5 activity. In some embodiments, a method of identifying a compound that decreases NLRC5 expression and/or NLRC5 activity is provided. The method comprises comparing the level of expression and/or activity of NLRC5 in the test cell compared to the level of expression and/or activity of NLRC5 in a cell, referred to as a control cell, that is the same type of cell and has not been contacted with the test compound, wherein if the level of expression and/or activity of NLRC5 in the test cell is less than the level of expression and/or activity in the control cell, the test compound is a compound that decreases NLRC5 expression and/or NLRC5 activity.

In some embodiments, the screening methods described herein further involve comparing the level of expression of MHC class I genes in the test cell to the level of expression in the control cell, wherein if the level of expression of MHC class I genes in the test cell is different from the level of expression in the control cell, the test compound is a compound that modulates MHC class I gene expression.

Pharmaceutical compositions that comprise an antibody that binds NLRC5 and a pharmaceutically acceptable carrier are provided. The antibody may inhibit NLRC5 expression and/or NLRC5 activity. In some embodiments, the pharmaceutical compositions that comprise such an antibody may be used for the treatment of a disease associated with aberrant expression of MHC class I genes.

In some embodiments, the method is a method to increase the efficacy and effectiveness of a vaccine by increasing NLRC5 expression and/or NLRC5 activity in a subject in need thereof. The method comprises administering to the subject a compound that increases NLRC5 expression and/or NLRC5 activity in an amount effective to increase MHC class I gene expression and increase the efficacy and effectiveness of the vaccine in the subject. Examples of compounds that may be used include, but are not limited to, siRNA, a synthetic organic chemical, a peptide, a natural fermentation product, and a substance extracted from a microorganism, plant, animal or cell culture medium.

### BRIEF DESCRIPTION OF THE DRAWINGS

FIG. 1 shows that NLRC5 contains an N-terminal bipartite NLS and can translocate into the nucleus. HEK293T cells were transfected with expression plasmids coding for GFP, or the indicated GFP fusion proteins. 48 hours post transfection, cells were treated with 10 nM leptomycin B (LMB) for 90 min, or left untreated. Fixed cells were stained with Hoechst 33342 to indicate the nuclei (scale bar: 10 μm). FIG. 1A shows the cellular localization of NLRC5 and CIITA upon LMB treatment. FIG. 1B shows the phylogenetic tree of CARD-containing NLRs. FIG. 1C is a schematic representation of the NLRC5 deletion mutant constructs used to map the nuclear localization signal. The position of the NLS is indicated by an asterisk. FIG. 1D shows the cellular localization of NLRC5 deletion mutants upon LMB treatment. FIG. 1E shows the sequence of the bipartite NLS found in the N-terminus of NLRC5. Alanine substitution of the right or left arm of the NLS was used to construct the NLSI and NLSII import mutant expression plasmids. FIG. 1F shows the cellular localization of the NLSI and NLSII mutant forms of NLRC5 upon LMB treatment.

FIG. 2 shows the induction of MHC class I and functionally related genes by NLRC5. RNA isolated from Jurkat T cells stably expressing the indicated GFP-fusion proteins was analyzed by qRT-PCR for the expression of the indicated genes; empty vector (GFP), wild-type NLRC5 (WT), Walker A mutant (A), Walker B mutant (B), Walker AB mutant (AB) (FIG. 2A). The same Jurkat T cell lines were examined for the expression of MHC class I heavy chain (HC), β2M, TAP1, and LMP2 by Western blot analysis. Actin levels are shown as a loading control (FIG. 2B). FIG. 2C shows the surface expression of MHC class I in Jurkat T cell lines expressing GFP (gray line) or the indicated GFP-NLRC5 fusion proteins (black line) examined by flow cytometry using anti-pan-MHC class I (HLA-A, -B, -C) and HLA-E antibodies. IFN-γ (100 U/ml) treatment was used as a positive control. Data obtained with an isotype control antibody is indicated by the shaded area. HEK293T cells were transiently transfected with the expression plasmids for GFP-fused to NLRC5 or CIITA (black line), or GFP only (gray line). The expression of MHC class I (HLA-A, -B, -C) or class II (HLA-DR) was analyzed by flow cytometry 48 hours post transfection. Data obtained with an isotype control antibody is indicated by the shaded area (FIG. 2D).

FIG. 3 shows that NLRC5 binds and transactivates MHC class I gene promoters. NLRC5-mediated transactivation of MHC class I and functionally related genes. HEK293T cells were transiently transfected with either expression vectors for GFP, GFP-NLRC5, or GFP-CIITA, along with luciferase reporter constructs of the indicated gene promoters. Cell lysates were analyzed 48 hours post transfection by dual-luciferase assay. Data are a representative of three independent experiments performed in duplicates, and error bars represent ±SD (FIG. 3A). FIG. 3B shows a schematic representation of the W/SXY module found in the promoters of MHC class I and class II genes. The position of the primers used in the ChIP assay are indicated with arrows (P1, P2). FIG. 3C shows NLRC5 occupancy, in terms of fold enrichment, at the HLA-A, -B or -DRA promoters, as determined by chromatin immunoprecipitation (ChIP). Jurkat T cells stably expressing the indicated GFP-fusion proteins were analyzed by ChIP assay using an anti-GFP antibody for immunoprecipitation and the indicated qPCR primers (B); empty vector (GFP), wild-type NLRC5 (WT), Walker A mutant (A), Walker B mutant (B), Walker AB mutant (AB). Error bars indicate standard error of the mean (±SEM) from three independent experiments.

FIG. 4 shows the knockdown of NLRC5 results in decreased upregulation of MHC class I upon IFN-γ treatment. HeLa cells were stimulated with IFN-γ (100 U/ml) for the indicated time points, and the kinetics of NLRC5, HLA-A and STAT1 expression were analyzed by qRT-PCR (FIG. 4A). HeLa cells were transfected with NLRC5-specific or control siRNAs. 16 hours post transfection, cells were stimulated with IFN-γ for 24 hours. Knockdown efficiency of NLRC5 was determined by qRT-PCR using gene specific primers and data were normalized to the expression of the GAPDH gene. Scr: control scrambled siRNA. Error bars represent the ±SD from one representative out of three independent experiments performed in duplicates. *p<0.05 (FIG. 4B). FIG. 4C shows the surface expression of MHC class I and β1-integrin analyzed by flow cytometry. FIG. 4D represents a model depicting the role of NLRC5 in the IFN-γ-induced upregulation of MHC class I genes.

FIG. 5 shows that NLRC5 import mutants do not enter the nucleus. Protein stability of GFP-NLRC5 wild-type and the indicated import mutants was verified by Western blot analysis using an anti-GFP antibody (FIG. 5A). FIG. 5B shows the quantification of the subcellular localization of wild-type NLRC5 and the indicated import mutants in transiently transfected HEK293T cells. 24 hours following transfection, cells were treated with 10 nM leptomycin B (LMB) for 90 min before fixing. Cells were observed with an epifluorescence microscope and counted as ‘cytosolic’ or ‘nuclear’ if the majority of the GFP signal was detected in the respective compartment, and ‘intermediate’ if the signal intensity in both compartments was comparable. Data was pooled from two independent experiments, performed in a blind controlled manner, and error bars represent ±SEM.

FIG. 6 shows the subcellular distribution of murine Nlrc5. HEK293T cells were transiently transfected with an expression plasmid encoding murine Nlrc5 fused to GFP. 48 hours post transfection cells were treated with 10 nM Leptomycin B (LMB) for 90 min or left untreated. The cells were fixed with 10% formaldehyde/PBS and stained with Hoechst 33342 to indicate the position of the nuclei (scale bar: 10 μm).

FIG. 7 shows a gene chip analysis reveals differential target gene expression between cells stably expressing wild-type and mutant forms of NLRC5. FIG. 7A is a schematic representation of the NBD mutant forms of NLRC5 that were stably expressed in Jurkat T cells. The Walker A mutant (K234A) is presumably defective in NTP binding, while the Walker B mutation (E311Q) is predicted to interfere with NTP hydrolysis. The Walker AB mutant harbors both mutations. FIG. 7B shows the hierarchical clustering of differentially expressed genes from Jurkat T cells stably expressing WT or mutant forms of NLRC5. Genes were considered significantly differentially expressed if their expression was 1.8 fold higher or lower in cells expressing the nonfunctional constructs (empty vector, A, or AB) as compared to cells expressing functional forms of NLRC5 (WT, B) with P<0.2. A heat-map is used to represent the RNA levels of selected genes from this list. Functional NLRC5-expressing Jurkat T cells show significantly higher expression of MHC class I and related genes involved in antigen presentation. The number of significant transcript clusters refers to the number of Affymetrix transcript clusters corresponding to the indicated gene that detected significantly different expression (see above). Fold change values use the average expression level in cells transfected with empty vector, A, or AB as a reference; thus, a positive fold change indicates higher gene expression (FIG. 7C).

FIG. 8 shows that NLRC5 does not activate NF-κB-, AP-1-, ISRE- or IRF3-dependent promoters, nor the promoters of IFN-α and IFN-β. HEK293T cells were transiently transfected with either empty vector (GFP), GFP-NLRC5, or GFP-CIITA expression plasmids, together with the indicated reporter plasmids. 48 hours post transfection, cell lysates were prepared and luciferase activity was measured by dual-luciferase assay. A reporter plasmid containing the HLA-A promoter was used as positive control. Data are a representative of three independent experiments performed in triplicates. Error bars represent ±SD.

FIG. 9 shows that NOD1, NOD2, and NLRC3 do not increase MHC class I expression in epithelial cells. HEK293T cells were transiently transfected with expression plasmids for the indicated GFP fusion proteins. The surface expression of MHC class I and class II was examined 48 hours post transfection by flow cytometry using anti-HLA-A, -B, -C or anti-HLR-DR antibodies by gating on GFP-positive cells. Data obtained with an isotype control antibody is indicated by the shaded area (FIG. 9A). HEK293T cells were transiently transfected with expression plasmids for the following GFP-fusion proteins: untransfected (---), empty vector (GFP), wild-type NLRC5 (WT), Walker A mutant (A), Walker B mutant (B), Walker AB mutant (AB), NOD1, NOD2, NLRC3. 48 hours post transfection, total cell lysates were prepared and Western blot analysis was performed with antibodies against the MHC class I heavy chain (HC) and GFP. An anti-tubulin antibody was used to demonstrate equal loading (FIG. 9B).

FIG. 10 shows that NLRC5 binds to MHC class I gene promoters in an epithelial cell line. Transiently transfected HEK293Tcells expressing the indicated GFP-fusion proteins were analyzed by chromatin immunoprecipitation (ChIP) assay using an anti-GFP antibody for immunoprecipitation and the corresponding promoter-specific qPCR primers. Promoter occupancy of the GFP-fusion proteins is given as fold enrichment at the HLA-A, -B or -GAPDH promoters. Error bars indicate standard error of the mean (±SEM) from four independent experiments.

FIG. 11 shows that MHC class I and functionally related genes are IFN-γ-inducible in Jurkat T cells and in HeLa cells. Jurkat T cells were stimulated with IFN-γ (100 U/ml) for the indicated time points and kinetics of NLRC5, HLA-A and STAT1 expression were analyzed by qRT-PCR (FIG. 11A). FIG. 11B shows western blot analysis of whole cell extracts obtained from Jurkat T cells stimulated for 16 hrs with IFN-γ (100 U/ml) or left untreated (−). HeLa cells were stimulated with IFN-γ (100 U/ml) for 0 (gray line) or 24 hrs (black line) and the surface expression of MHC class I was analyzed by flow cytometry using an anti-HLA-A, -B, -C antibody. Data obtained with an isotype control antibody is indicated by the shaded area (FIG. 11C).

FIG. 12 shows the knockdown of NLRC5 results in a decreased upregulation of MHC class I expression upon IFN-γ treatment while MHC class II and CIITA induction remain unaffected. HeLa cells were transiently transfected with two different siRNAs targeting NLRC5 (#1, #2) or control siRNAs. 16 hours post transfection, cells were stimulated with IFN-γ (100 U/ml) for 24 hours. Knockdown efficiency of HLA-B, CIITA, and HLA-DR were determined by qRT-PCR using gene specific primers, and data were normalized to the expression of the GAPDH gene. Scr: control scrambled siRNA. Error bars represent ±SD from a representative experiment out of a total of three independent experiments performed in duplicates. *p<0.05.

### DETAILED DESCRIPTION OF THE INVENTION

Described herein is the identification of a molecule, NOD-like receptor family CARD domain containing 5 (NLRC5; also called NOD27, CLR16.1; NCBI reference sequence NM_{—}032206) that regulates all MHC Class I genes. NLRC5 represents an excellent target to augment or repress MHC class I-mediated immune responses.

MHC class I molecules are composed of MHC-encoded heavy chains and the invariant subunit β2-microglobulin (β2M) (1). Humans have three classical MHC class Ia molecules (HLA-A, HLA-B and HLA-C), which are vital to the detection and elimination of viruses, cancerous cells and transplanted cells. In addition, there are three non-classical MHC class Ib molecules (HLA-E, HLA-F and HLA-G), which have immune regulatory functions (2, 3). Antigen-derived peptides are presented by MHC class I-β2M complexes at the cell surface to CD8 T cells carrying an antigen-specific T cell receptor. Peptides are mostly produced from the degradation of cytoplasmic proteins by a specialized proteasome, or “immunoproteasome”, which is optimized to generate MHC class I peptides and contains several IFN-γ-inducible subunits, such as LMP2 and LMP7 (4). Peptide loading onto MHC class I is carried out by the peptide loading complex (PLC), which includes the MHC class I heavy chain, β2M, tapasin, ERp57, calreticulin and TAP1/TAP2, a transporter that translocates peptides from the cytoplasm into the ER (4, 5).

Unlike MHC class II, which is found mainly in antigen-presenting cells, MHC class Ia is ubiquitously expressed in almost all nucleated cells (1, 6). Both MHC class I and class II genes are highly inducible by IFN-γ stimulation and share similar cis-regulatory elements in their promoters, termed W/S, X1, X2 and Y-box motifs, which also associate with similar transcription factor complexes (7, 8). These transcription factors include the X-box binding trimeric RFX protein complex (composed of RFX5, RFXAP and RFXANK), the X2-box binding CREB/ATF, and the Y-box binding NF-Y protein (composed of NF-YA, NF-YB and NF-YC) (9). Together they form a macromolecular nucleoprotein complex called the MHC enhanceosome (10).

CIITA, a member of the NLR or nucleotide binding domain (NBD), leucine rich repeat (LRR) family of proteins (11, 12), regulates the transcription of MHC class II by associating with the MHC enhanceosome (10, 13). The expression of CIITA is induced in B cells and dendritic cells as a function of developmental stage and is inducible by IFN-γ in most cell types (14-16). There are 22 NLR proteins in humans, which share three characteristic functional domains: an N-terminal protein-protein interaction domain such as a CARD or a PYRIN, a centrally located NBD (or NACHT) and C-terminal LRRs (11, 12). Aside from CIITA, NLR proteins are localized in the cytoplasm and contribute to the innate immune response by recognizing microbial products and exogenous danger signals, leading to inflammation and/or cell death (11, 12).

Previous studies have shown that CIITA also has a role in the transactivation of MHC class I genes, although to a lesser extent than the role it plays in regulation of MHC class II (6-9, 17). The expression of CIITA is generally restricted to lymphocytes and professional antigen-presenting cells, and is thus unlikely to account for the ubiquitous expression of MHC class I (6, 18). Furthermore, while mutations of the CIITA gene can cause bare lymphocyte syndrome (BLS), an immunodeficiency characterized by the lack of MHC class II expression, a subgroup of BLS patients that lack CIITA retains the expression of MHC class I but not MHC class II (19, 20). Similarly, in mice deficient for CIITA, both constitutive and IFN-γ-induced expression of MHC class I molecules is intact (21-23). These findings indicate that, in addition to CIITA, other molecules or mechanisms are involved in the regulation of MHC class I expression.

Accordingly, the present methods and compositions make it possible to modulate the expression of MHC class I proteins by modulating a NLR protein, NLRC5 (NOD27/CLR16.1). Similar to CIITA, NLRC5 is highly inducible by IFN-γ and can translocate into the nucleus. NLRC5 was shown to activate the promoters of MHC class I genes and induce the transcription of MHC class I, as well as related genes involved in MHC class I antigen presentation. The methods described herein are useful to treat subjects in need of treatment of or protection against diseases or conditions associated with aberrant expression of MHC class I genes. As used herein, the subject is an animal, typically a mammal, such as a dog, a cat, a horse, a sheep, a goat, a cow or a rodent. In specific embodiments, the mammal is a human.

Accordingly, in some embodiments, methods to increase or decrease MHC class I expression by increasing or decreasing NLRC5 expression and/or activity are provided. In some embodiments, the catalytic activity of NLRC5 is targeted leading to an increase or decrease in the activity of NLRC5. The examples disclosed below describe a NLRC5 catalytically inactive mutant (Walker A mutant) and a catalytically active mutant (Walker B mutant). These and other regions of NLRC5 may be targeted to modulate the activity of NLRC5. As used herein, modulate and modulation means to change the normal expression and/or activity of a protein. Modulation includes an increase in the expression and/or activity (upregulation or agonist activity) and a decrease in the expression and/or activity (downregulation or inhibition). MHC class I molecules include, but are not limited to, the classical (class 1a) MHC I molecules (HLA-A, -B, -C), other non-classical (class Ib) MHC Class I molecules (HLA-E, -F, -G), and β2-microglobulin. MHC Class I molecules include human MHC Class I molecules (the human leukocyte antigen (HLA) complex) and vertebrate equivalents thereof, such as class I antigens of the H-2 locus of mice, in particular H-2 D and K. There are also numerous MHC class I-like genes, many of which are coded outside of the canonical MHC Class I region, including HFE, MICA, MICE, CD1-a, -b, -c, -d, and members of the ULPB family.

The compounds that are used to modulate the expression of MHC class I proteins by modulating NLRC5 expression include, but are not limited to, antibodies, short-interfering RNAs (siRNAs), a synthetic organic chemical, a peptide, a natural fermentation product, and a substance extracted from a microorganism, plant, animal or cell culture medium.

The term-antibody as used herein refers to all types of immunoglobulins, including IgG, IgM, IgA, IgD, and IgE. The term “immunoglobulin” includes the subtypes of these immunoglobulins, such as IgG1: IgG2, IgG3, IgG4, etc. An antibody may be of any species of origin, including (for example) mouse, rat, rabbit, horse, or human, or may be chimeric antibodies. See, e.g., M. Walker et al., Molec. Immunol. 26, 403-11 (1989). An antibody can be polyclonal or monoclonal. The term “antibody” as used herein also includes antibody fragments that bind a target antigen. These include, for example, Fab, F(ab′)2, and Fv fragments. Such fragments can be produced by known techniques. The term “polyclonal antibody” as used herein refers to multiple immunoglobulins in antiserum produced to an antigen following immunization, and which may recognize and bind to one or more epitopes to that antigen. Polyclonal antibodies can be produced by immunizing a suitable subject of any species of origin, including (for example) mouse, rat, rabbit, goat, sheep, chicken, donkey, horse or human, with an antigen to which a monoclonal antibody to the target binds, collecting immune serum from the animal, and separating the polyclonal antibodies from the immune serum, in accordance with known procedures. The term “antibody” as used herein also refers to a monoclonal antibodies. The monoclonal antibodies may be recombinant monoclonal antibodies produced according to known methods, such as the methods disclosed in Reading, U.S. Pat. No. 4,474,893, or Cabilly et al., U.S. Pat. No. 4,816,567. The antibodies may also be chemically constructed by specific antibodies made according to the method disclosed in Segel et al., U.S. Pat. No. 4,676,980. Applicants specifically intend that the disclosure of all U.S. patent references cited herein be incorporated herein by reference in their entirety.

Examples of epitopes used to generate antibodies include, but are not limited to the following sequences:

(SEQ ID NO: 1; mouse NLRC5 epitope)
MAARQHSPLLMDAESIRLNNENLWAWLVRLLSKNPEWLSAKLRSFLPTM
DLDCSYEPSNEVIHRQLNRLFAQGMATWKSFINDLCFELDVPLDMEIPL
VSIWGPRDEFSKQLGAGEECPGPQLYHGAKRPFQSYGSSPRRKNSKKQQ
LELAKKYLKLLKTSAQQWHGGVCPGAWLTHSPQTYIPPVLQWSRATAPL
DAQEGATLGDPEAADNIDVSI;
(SEQ ID NO: 2; human NLRC5 epitope)
MDPVGLQLGNKNLWSCLVRLLTKDPEWLNAKMKFFLPNTDLDSRNETLD
EQRVILQLNKLHVQGSDTWQSFIFICVCMQLEVPLDLEVLLLSTFGYDD
GFTSQLGAEGKQPESQLHHGLKRPHQSCGSSPRRKQCKKQQLELAKKYL
QLLRTSAQQRYRSQIPGSGQPAFHQVYVPPILRRATASLDTPEGAIMGD
VKVEDGADVSI.

In some embodiments, the antibodies are generated using any region of the NLRC5 sequence provided below:

Mouse NLRC5 full length
(SEQ ID NO: 3)
MDAESIRLNNENLWAWLVRLLSKNPEWLSAKLRSFLPTMDLDCSYEPSNPEVIHRQL
NRLFAQGMATWKSFINDLCFELDVPLDMEIPLVSIWGPRDEFSKQLGAGEESCPGPQ
LYHGAKRPFQSYGSSPRRKNSKKQQLELAKKYLKLLKTSAQQWHGGVCPGAWLTP
HSPQTYIPPVLQWSRATAPLDAQEGATLGDPEAADNIDVSIQDLFSFKAHKGPRVTV
LLGKAGMGKTTLAYRLRWRWAQGQLDRFQALFLFEFRQLNMITQLPTLPQLLFDLY
LMPESEPDAVFQYLKENAQEVLLIFDGLDEALHADSVGTDNAGSALTLFSELCHGNL
LPGCWVMTTSRPGKLPSCVPTEAATVHMWGFDGLRVEKYVTCFFSDLLSQELALKE
MRTNARLRGMCAIPALCTVTCFCLRRLLPGSSPGQSAALLPTITQLYLQMVETFSPSE
TLLDTSILGFGKVALRGLDTGKVVFSVEDISPQLMSFGAVHSLLTSFCIHTRPGHEEIG
YAFVHLSLQEFFAALYLMASHTVDKDTLVEYVTLNSHWVLRTKGRLGLSDHLPAFL
AGLASHTCHMFLCQLAQQDRAWVGSRQAAVIQVLRKLASRKLTGPKMIELYHCVA
ETQDLELARFTAQSLPSRLSFHNFPLTHADLAALANILEHRDDPIHLDFDGCPLEPHCP
EALVGCGQVENLSFKSRKCGDAFAEALCRSLPTMGSLKTLGLTGSRITAQGISHLIQT
LPLCSQLEEVSLHDNQLKDPEVLSLVELLPSLPKLQKLDLSRNSFSRSILLSLVKVAIT
CPTVRKLQVRELDLIFYLSPVTETATQQSGASDVQGKDSLKEGQSRSLQLRLQKCQL
RIRDAEALVELFQKSPQLEEVNLSGNHLEDDGCRLVAEAASQLHIAQKLDLSDNGLS
QTGVTYVLKAMSTCGTLEDLHISLLNNTVVLTFAQEPREQEGSCKGRAPLISFVSPVT
SELSQRSRRIRLTHCGFLAKHTETLCEALRASCQTHNLDHLDLSDNSLGGKGVILLTE
LLPGLGPLKSLNLSRNGLSMDAVFSLVQCLSSLQWVFHLDVSLESDCIFLRGAGTSR
DALEPKFQTGVQVLELSQRYTSRSFCLQECQLEPTSLTFLCATLEKSPGPLEVQLSCK
SLSDDSLKILLQCLPQLPQLSLLQLRHTVLSSRSPFLLADIFNLCPRVRKVTLRSLCHA
VLHFDSNEEQEGVCCGFPGCSLSQEHMETLCCALSKCNALSQLDLTDNLLGDIGLRC
LLECLPQLPISGWLDLSHNNISQEGILYLLETLPSYPNIQEVSVSLSSEQIFRMCFSKKE
GAGTSLRLCECSFSPEQVSKLASSLSQAQQLTELWLTKCHLDLPQLTMLLNLVNRPT
GLLGLRLEEPWVDSVSLPALMEVCAQASGCLTELSISEIQRKLWLQLEFPHQEGNSDS
MALRLAHCDLETEHSHLMIQLVETYARLQQLSLSQVSFNDNDGTSSKLLQNILLSSCE
LKSFRLTFSQVSTKSLTHLAFGLGHCHHLEELDFSNNSLREEDTELLMGALQGTCRL
KKLHLSFLPLGASSLALLIQGLSRMTLLQDLCLSHNQIGDVGTQCLAAILPKLPELRKF
DLSHNQIGDVGTQCLAAILPKLPELRKFNLSHNQIGHVGTQCLAAILPKLPELRKFDL
SRNQIGDVGTQCLAAILPKLPELRKFDLSGNRIGPAGGVQLVKSLTHFEHLEEIKLGN
NALGEPTALELAQRLPPQLRVLCLPSSHLGPEGALGLAQALEQCPHIEEVSLAENNLA
GGVPRFSKRLPLLRQIDLEFCKIEDQAARHLAANLTLFPALEKLLLSGNLLGDEVAAE
LAQVLPQMGQLKKVNLEWNRITARGAQLLAQGLVQGSCVPVIRLWNNPILNDVAQS
LQSQEPRLDFSITDQQTL
Human NLRC5 full length
(SEQ ID NO: 4)
MDPVGLQLGNKNLWSCLVRLLTKDPEWLNAKMKFFLPNTDLDSRNETLDPEQRVIL
QLNKLHVQGSDTWQSFIHCVCMQLEVPLDLEVLLLSTFGYDDGFTSQLGAEGKSQPE
SQLHHGLKRPHQSCGSSPRRKQCKKQQLELAKKYLQLLRTSAQQRYRSQIPGSGQPH
AFHQVYVPPILRRATASLDTPEGAIMGDVKVEDGADVSISDLFNTRVNKGPRVTVLL
GKAGMGKTTLAHRLCQKWAEGHLNCFQALFLFEFRQLNLITRFLTPSELLFDLYLSP
ESDHDTVFQYLEKNADQVLLIFDGLDEALQPMGPDGPGPVLTLFSHLCNGTLLPGCR
VMATSRPGKLPACLPAEAAMVHMLGFDGPRVEEYVNHFFSAQPSREGALVELQTNG
RLRSLCAVPALCQVACLCLHHLLPDHAPGQSVALLPNMTQLYMQMVLALSPPGHLP
TSSLLDLGEVALRGLETGKVIFYAKDIAPPLIAFGATHSLLTSFCVCTGPGHQQTGYA
FTHLSLQEFLAALHLMASPKVNKDTLTQYVTLHSRWVQRTKARLGLSDHLPTFLAG
LASCTCRPFLSHLAQGNEDCVGAKQAAVVQVLKKLATRKLTGPKVVELCHCVDET
QEPELASLTAQSLPYQLPFHNFPLTCTDLATLTNILEHREAPIHLDFDGCPLEPHCPEA
LVGCGQIENLSFKSRKCGDAFAEALSRSLPTMGRLQMLGLAGSKITARGISHLVKAL
PLCPQLKEVSFRDNQLSDQVVLNIVEVLPHLPRLRKLDLSSNSICVSTLLCLARVAVT
CPTVRMLQAREADLIFLLSPPTETTAELQRAPDLQESDGQRKGAQSRSLTLRLQKCQ
LQVHDAEALIALLQEGPHLEEVDLSGNQLEDEGCRLMAEAASQLHIARKLDLSDNGL
SVAGVHCVLRAVSACWTLAELHISLQHKTVIFMFAQEPEEQKGPQERAAFLDSLML
QMPSELPLSSRRMRLTHCGLQEKHLEQLCKALGGSCHLGHLHLDFSGNALGDEGAA
RLAQLLPGLGALQSLNLSENGLSLDAVLGLVRCFSTLQWLFRLDISFESQHILLRGDK
TSRDMWATGSLPDFPAAAKFLGFRQRCIPRSLCLSECPLEPPSLTRLCATLKDCPGPL
ELQLSCEFLSDQSLETLLDCLPQLPQLSLLQLSQTGLSPKSPFLLANTLSLCPRVKKVD
LRSLHHATLHFRSNEEEEGVCCGRFTGCSLSQEHVESLCWLLSKCKDLSQVDLSANL
LGDSGLRCLLECLPQVPISGLLDLSHNSISQESALYLLETLPSCPRVREASVNLGSEQS
FRIHFSREDQAGKTLRLSECSFRPEHVSRLATGLSKSLQLTELTLTQCCLGQKQLAILL
SLVGRPAGLFSLRVQEPWADRARVLSLLEVCAQASGSVTEISISETQQQLCVQLEFPR
QEENPEAVALRLAHCDLGAHHSLLVGQLMETCARLQQLSLSQVNLCEDDDASSLLL
QSLLLSLSELKTFRLTSSCVSTEGLAHLASGLGHCHHLEELDLSNNQFDEEGTKALMR
ALEGKWMLKRLDLSHLLLNSSTLALLTHRLSQMTCLQSLRLNRNSIGDVGCCHLSEA
LRAATSLEELDLSHNQIGDAGVQHLATILPGLPELRKIDLSGNSISSAGGVQLAESLVL
CRRLEELMLGCNALGDPTALGLAQELPQHLRVLHLPFSHLGPGGALSLAQALDGSPH
LEEISLAENNLAGGVLRFCMELPLLRQIDLVSCKIDNQTAKLLTSSFTSCPALEVILLS
WNLLGDEAAAELAQVLPQMGRLKRVDLEKNQITALGAWLLAEGLAQGSSIQVIRL
WNNPIPCDMAQHLKSQEPRLDFAFFDNQPQAPWGT

In some embodiments, pharmaceutical compositions comprising an antibody that binds NLRC5 and a pharmaceutical acceptable carrier are provided.

The term “short-interfering RNAs (siRNA)” refers to small double-stranded RNAs that interfere with gene expression. siRNAs are an intermediate of RNA interference, the process by which double-stranded RNA silences homologous genes. siRNAs, are typically comprised of two single stranded RNAs, of about 21 nucleotides long that form a 19 base pair duplex with about 2 nucleotide 3′ overhangs. Processing of the double stranded RNA by an enzymatic complex, for example polymerases, results in cleavage of the double stranded RNA to produce siRNAs. The antisense strand of the siRNA is used by an RNA interference (RNAi) silencing complex to guide mRNA cleavage, so promoting mRNA degradation. To silence a specific gene using siRNAs, for example in a mammalian cell, the base pairing region is selected to avoid chance complementarity to an unrelated mRNA.

In some embodiments, methods to reduce viral infection by increasing NLRC5 expression and/or NLRC5 activity in a subject in need thereof are provided. The method comprises administering to the subject a compound that increases NLRC5 expression and/or activity. The compound is administered in an amount to effective to increase NLRC5 expression and/or NLRC5 activity which boosts MHC class I expression and reduces the viral infection in the subject. A subject in need thereof already has a viral infection or is at risk of having a viral infection. Risk factors for a viral infection include: immunosuppression, immunocompromise, age, trauma, burns (e.g., thermal burns), surgery, foreign bodies, cancer, newborns especially newborns born prematurely. In some embodiments, the expression and/or activity of NLRC5 is increased by at least approximately 10% relative to normal. In some embodiments, the expression of NLRC5 is increased by at least approximately 20%, 30%, 40%, 50%, 60%, 70%, 80%, 90%, 95%, or 99% relative to normal.

Examples of viruses include but are not limited to: Retroviruses, human immunodeficiency viruses including HIV-1, HDTV-III, LAVE, HTLV-III/LAV, HIV-III, HIV-LP, Cytomegaloviruses (CMV), Picornaviruses, polio viruses, hepatitis A virus, enteroviruses, human Coxsackie viruses, rhinoviruses, echoviruses, Calciviruses, Togaviruses, equine encephalitis viruses, rubella viruses, Flaviruses, dengue viruses, encephalitis viruses, yellow fever viruses, Coronaviruses, Rhabdoviruses, vesicular stomatitis viruses, rabies viruses, Filoviruses, ebola virus, Paramyxoviruses, parainfluenza viruses, mumps virus, measles virus, respiratory syncytial virus (RSV), Orthomyxoviruses, influenza viruses, Bungaviruses, Hantaan viruses, phleboviruses and Nairo viruses, Arena viruses, hemorrhagic fever viruses, reoviruses, orbiviruses, rotaviruses, Birnaviruses, Hepadnaviruses, Hepatitis B virus, parvoviruses, Papovaviridae, papilloma viruses, polyoma viruses, Adenoviruses, Herpesviruses including herpes simplex virus 1 and 2, varicella zoster virus, Poxviruses, variola viruses, vaccinia viruses, Irido viruses, African swine fever virus, delta hepatitis virus, non-A, non-B hepatitis virus, Hepatitis C, Norwalk viruses, astroviruses, and unclassified viruses.

In some embodiments, methods to inhibit cancer by increasing NLRC5 expression and/or NLRC5 activity in a subject are provided. The method comprises administering to the subject a compound that increases NLRC5 expression and/or activity. The compound is administered in an amount effective to increase NLRC5 expression and/or NLRC5 activity to an extent sufficient to boost MHC class I expression and inhibit cancer (prevent the occurrence or re-occurrence of cancer, reduce the extent to which cancer occurs, reverse cancer that has already occurred) in the subject. In some embodiments, the expression and/or activity of NLRC5 is increased by at least approximately 10% relative to normal. In some embodiments, the expression of NLRC5 is increased by at least approximately 20%, 30%, 40%, 50%, 60%, 70%, 80%, 90%, 95%, or 99% relative to normal.

Examples of cancer include but are not limited to, carcinoma, including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and nonHodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

In some embodiments, methods to inhibit tissue or organ rejection by decreasing NLRC5 expression and/or NLRC5 activity in a subject are provided. The method comprises administering to the subject a compound that decreases NLRC5 expression and/or activity. The compound is administered in an amount effective to decrease NLRC5 expression and/or NLRC5 activity which inhibits MHC class I expression and inhibits tissue or organ expression in the subject. In some embodiments, the expression and/or activity of NLRC5 is decreased by at least approximately 10% relative to normal. In some embodiments, the expression of NLRC5 is decreased by at least approximately 20%, 30%, 40%, 50%, 60%, 70%, 80%, 90%, 95%, or 99% relative to normal. In some embodiments, the compounds of the invention are used to treat graft-versus-host diseases (GVHD). GVHD are a common complication of allogeneic bone marrow transplantation in which functional immune cells in the transplanted marrow recognize the recipient as “foreign” and mount an immunologic attack.

In some embodiments, methods to increase the efficacy and effectiveness of a vaccine by increasing NLRC5 expression and/or NLRC5 activity in a subject in need thereof are provided. Extracellular antigens (including vaccines) can be processed in dendritic cells and presented to CD8 T cells using MHC class I molecules. This process is called cross-presentation. The methods comprise administering to the subject a compound that increases NLRC5 expression and/or NLRC5 activity in an amount effective to increase MHC class I gene expression and increase the efficacy and effectiveness of the vaccine in the subject. Examples of compounds that may be used include, but are not limited to, siRNA, a synthetic organic chemical, a peptide, a natural fermentation product, and a substance extracted from a microorganism, plant, animal or cell culture medium. The vaccines may be useful to treat and/or inhibit various diseases including, but not limited to, cancer and viral infections.

In some embodiments, compounds modulating NLRC5 expression and/or NLRC5 activity are administered in combination with compounds that modulate CIITA expression and/or CIITA activity. Without being bound by theory, it is postulated that since CIITA also plays a role in transactivating MHC class I genes, compounds that modulate CIITA expression and/or CIITA activity will also modulate MHC class I gene expression. In addition, administration of compounds modulating NLRC5 expression and/or NLRC5 activity in combination with compounds that modulate CIITA expression and/or CIITA activity results in modulation of both MHC class I and MHC class II molecules, which are also involved in various pathologic conditions including cancer, autoimmune diseases, transplanted organ rejections. Most transplanted tissues express MHC class I, but not class II molecules. However, some transplanted tissues, especially of hematopoietic cell origin express MHC class II in addition to MHC class I molecules. Compounds that may be used to modulate CIITA expression and/or CIITA activity include, but are not limited to, (siRNAs), a synthetic organic chemical, a peptide, a natural fermentation product, and a substance extracted from a microorganism, plant, animal or cell culture medium. In some embodiments, the expression of CIITA is modulated by at least approximately 10%, 20%, 30%, 40%, 50%, 60%, 70%, 80%, 90%, 95%, or 99% relative to normal.

The compounds described herein are administered in effective amounts. An effective amount is a dose sufficient to provide a medically desirable result and can be determined by one of skill in the art using routine methods. In the treatment of diseases associated with a aberrant expression of MHC class I genes, an effective amount will be that amount necessary to modulate NLRC5 expression and/or NLRC5 activity. In some embodiments, an effective amount is an amount which results in any improvement in the condition being treated. In some embodiments, an effective amount may depend on the type and extent of disease or condition being treated and/or use of one or more additional therapeutic agents. However, one of skill in the art can determine appropriate doses and ranges of compounds to use, for example based on in vitro and/or in vivo testing and/or other knowledge of compound dosages.

When administered to a subject, effective amounts will depend, of course, on the particular disease being treated; the severity of the disease; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

An effective amount typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations, for one or several days (depending of course of the mode of administration and the factors discussed above).

Actual dosage levels can be varied to obtain an amount that is effective to achieve the desired therapeutic response for a particular patient, compositions, and mode of administration. The selected dosage level depends upon the activity of the particular compound, the route of administration, the severity of the radiation exposure, the tissue being treated, and prior medical history of the patient being treated. However, it is within the skill of the art to start doses of the compound at levels lower than required to achieve the desired therapeutic effort and to gradually increase the dosage until the desired effect is achieved.

Screening methods to identify compounds that modulate NLRC5 expression and/or NLRC5 activity are also provided. The method comprises contacting a test cell with a test compound, wherein the cell comprises a NLRC5 nucleic acid, and comparing the level of expression and/or activity of NLRC5 in the test cell to the level of expression and/or activity of NLRC5 in a cell, referred to as a control cell, that has not been contacted with the test compound. The test compound is identified as a compound that modulates NLRC5 expression and/or activity if the level of expression and/or activity of NLRC5 is changed as compared to its expression and/or activity in the control cell. The screening methods are carried out under conditions under which NLRC5 is expressed. Examples of cells that can be screen compounds include, but are not limited to, human embryonic kidney 293T (HEK293T) cells, Jurkat T cells, and HeLa cells. Such screening for molecules that modulate NLRC5 expression and/or activity can easily be performed on a large scale, e.g., by screening candidate compounds from libraries of synthetic and/or natural molecules. In some embodiments, the screening methods further comprise comparing the level of expression of MHC class I genes in the test cell to the level of expression in the control cell, wherein if the level of expression of MHC class I genes in the test cell is changed as compared to the level of expression in the control cell, the test compound is a compound that also modulates MHC class I gene expression.

The compounds modulating NLRC5 expression and pharmaceutical compositions containing these compounds are administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain. In some embodiments, the compositions may be administered systemically.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the drug, it is desirable to slow the absorption of the drug from a subcutaneous or intramuscular injection. This result can be accomplished by the use of a liquid suspension of amorphous materials with poor water solubility. Delayed absorption of a parenterally administered drug also is accomplished by dissolving or suspending the drug in an oil vehicle Likewise, injectable depot forms are made by forming microencapsule matrices of the drug in biodegradable polymers such a polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissue.

In one embodiment, the method is one comprising oral administration of a pharmaceutical composition described herein. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556).

In such solid dosage forms, the active compound is mixed with, or chemically modified to include, at least one inert, pharmaceutically acceptable excipient or carrier. The excipient or carrier may permit increased uptake of the compound, overall stability of the compound and/or circulation time of the compound in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropyhnethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffm, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

The solid dosage forms of tablets, dragees, capsules, pills, and granules can be prepared with coatings and shells, such as enteric coatings and other coatings well known in the pharmaceutical formulating art. Liquid dosage forms for oral administration include pharmaceutically acceptable emulsions, solutions, suspensions, syrups, and elixirs. In addition to the active compounds, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Compounds described herein can also be administered via pulmonary delivery. The compound is delivered to the lungs of a mammal, such as a mammal that is inhaling. Contemplated for use in the present methods are a wide range of mechanical devices designed for pulmonary delivery of therapeutic products, including, but not limited to, nebulizers, metered dose inhalers, and powder inhalers, all of which are familiar to those skilled in the art. All such devices require the use of formulations suitable for the dispensing of a compound of the invention. Typically, each formulation is specific to the type of device employed and can involve the use of an appropriate propellant material, in addition to diluents, adjuvants, and/or carriers useful in therapy.

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

### EXAMPLES

### Materials and methods

### Cell Lines and Reagents.

Human embryonic kidney 293T (HEK293T) cells (ATCC#: CRL-11268) and HeLa cells (ATCC#: CCL-2) were cultured in Dulbecco's modified eagle medium (DMEM) supplemented with 10% fetal bovine serum (FBS) and penicillin (100 U/ml)/streptomycin (100 μg/ml, Gibco). Jurkat T cells (ATCC#: TIB 152) were maintained in RPMI-1640 (Thermo Scientific) supplemented with 10% FBS and penicillin/streptomycin. HEK293T were transiently transfected using FuGENE 6 Transfection Reagent (Roche) in serum-free media, according to the manufacturer's protocol. Recombinant human IFN-γ is from BioLegend. Leptomycin B (LMB) was obtained from LC Laboratories.

### Flow Cytometry.

Antibodies against human HLA-A, -B, -C (W6/32), HLA-E (3D12), HLA-DR (L243, all from Biolegend) and β1-integrin (TS2/16, a kind gift from Dr. Martin Hemler) were used in this study. Cells were stained, washed, and resuspended in PBS/1% FBS/0.05% NaN₃, and analyzed by FACSCalibur (Becton Dickinson) followed by analysis using FlowJo software.

### Knockdown of NLRC5 by RNA Interference.

HeLa cells (0.5×10⁶/well) were transfected with 20 nM siRNA using Hyperfect (Qiagen) according to the manufacturer's instructions. Cells were stimulated 16 hrs post transfection with 100 U/ml IFN-γ (BioLegend). After 24 hrs stimulation, cells were harvested and analysed by flow cytometry and quantitative real-time PCR. The control siRNA (scrambled), as well as siRNAs targeting NLRC5 were obtained from Ambion.

### Luciferase Assay.

HEK293T cells were split into 24-wells and co-transfected with 300 ng of either GFP, GFP-NLRC5 or GFP-CIITA expression plasmids and 100 ng of the indicated luciferase reporter constructs. 50 ng per well of promoterless Renilla luciferase vector (pRL-null, Promega) was included for normalization of transfection efficiency. Cells were harvested 48 hrs post transfection, and cell lysates were analysed using the Dual-Luciferase Reporter Assay System (Promega), according to the manufacturer's instructions. The reporter gene constructs were previously described (31).

### Statistical Analysis.

Data were subjected to Student's t test for analysis of statistical significance, and a P-value of <0.05 was considered to be significant.

### Cloning of Human and Murine NLRC5 and Construction of Expression Plasmids.

Full-length human NLRC5 and deletion mutants were cloned into a modified pcDNA3.1-based expression vector containing GFP, by standard cloning techniques. The full-length cDNA encoding human NLRC5 was obtained from the following cDNA clones: COL10077, SMINT2013032, IMAGE4152674, and confirmed by DNA sequencing. A deviation from the NLRC5 reference sequence (NM_{—}032206) was corrected using the following primer pair:

L191P fwd
SEQ ID NO: 5)
5′-CACAGCATCCTTAGACACTCCGGAGGGGGCCATTATGG-3′
L191P rev
(SEQ ID NO: 6)
5′-CCATAATGGCCCCCTCCGGAGTGTCTAAGGATGCTGTGG-3′

For the PCR amplification of the full-length cDNA, the following primers were used:

NLRC5 fwd
(SEQ ID NO: 7)
5′-ATATAGATCTGACCCCGTTGGCCTCCAG-3′
NLRC5 rev
(SEQ ID NO: 8)
5′-ATATTCTAGATCAAGTACCCCAAGGGGCCTG-3′

For the generation of deletion mutants, the following primers were used:

CARD fwd
(SEQ ID NO: 9)
5′-ATATAGATCTGACCCCGTTGGCCTCCAG-3′
CARD rev
(SEQ ID NO: 10)
5′-ATATGAATTCTTAGCCCTTGTTAACCCTGGTGTTGAAG-3′
ΔCARD fwd
(SEQ ID NO: 11)
5′-ATATAGATCTGAGTTGGCCAAGAAGTAC-3′
ΔCARD rev
(SEQ ID NO: 12)
5′-ATATTCTAGATTAAGTACCCCAAGGGGCCTG-3′
NACHT fwd
(SEQ ID NO: 13)
5′-ATATAGATCTGAGTTGGCCAAGAAGTAC-3′
NACHT rev
(SEQ ID NO: 14)
5′-ATATTCTAGATTAGCTGAGATTCTCTATCTG-3′
LRR fwd
(SEQ ID NO: 15)
5′-ATATAGATCTTTTAAGAGCAGGAAGTGTG-3′
LRR rev
(SEQ ID NO: 16)
5′-ATATTCTAGATTAAGTACCCCAAGGGGCCTG-3′
ΔLRR fwd
(SEQ ID NO: 17)
5′-ATATAGATCTGACCCCGTTGGCCTCCAG-3′
ΔLRR rev
(SEQ ID NO: 18)
5′-ATATTCTAGATTAGCTGAGATTCTCTATCTG-3′

Point mutations were introduced using the QuikChange Site-Directed Mutagenesis Kit (Stratagene) on an N-terminal fragment of NLRC5, using the following primers:

NLSI (RRK132/133/134A)
fwd
(SEQ ID NO: 19)
5′-GAGCTGTGGGTCCTCACCCGCCGCGGCGCAGTGCAAGAAGCAGC
AG-3′
rev
(SEQ ID NO: 20)
5′-CTGCTGCTTCTTGCACTGCGCCGCGGCGGGTGAGGACCCACAGC
TC-3′
NLSII (KR121/122A)
fwd
(SEQ ID NO: 21)
5′-CAGCTCCACCATGGCCTGGCGGCCCCACATCAGAGCTGTGG-3′
rev
(SEQ ID NO: 22)
5′-CCACAGCTCTGATGTGGGGCCGCCAGGCCATGGTGGAGCTG-3′
Walker A (K234A)
fwd
(SEQ ID NO: 23)
5′-GGAAGGCTGGCATGGGCGCGACCACGCTGGCCCACCG-3′
rev
(SEQ ID NO: 24)
5′-CGGTGGGCCAGCGTGGTCGCGCCCATGCCAGCCTTCC-3′
Walker B (E311Q)
fwd
(SEQ ID NO: 25)
5′-GATCTTTGATGGGCTAGATCAGGCCCTCCAGCCTATGGGTCC-3′
rev
(SEQ ID NO: 26)
5′-GGACCCATAGGCTGGAGGGCCTGATCTAGCCCATCAAAGATC-3′

The mutated N-terminal fragments were confirmed by DNA sequencing and subsequently reinserted into a plasmid containing the full-length cDNA of NLRC5 fused to GFP.

Murine Nlrc5 was amplified from spleen-derived cDNA from a C57BL/6 mouse and cloned into the GFP-pcDNA3.1 expression vector using the following primers:

murine Nlrc5 fwd
(SEQ ID NO: 27)
5′-ATATGGATCCATGGACGCTGAGAGCATCCGACTG-3′
murine Nlrc5 rev
(SEQ ID NO: 28)
5′-ATATATCTAGATCAAAGAGTCTGCTGGTCAGTG-3′

The GFP-CIITA expression plasmid was constructed by subcloning the cDNA of the human B-cell form of CIITA into the EcoRI/XhoI sites of the GFP expression vector described above.

### Generation of Stable Jurkat T Cell Lines.

Stable cell lines were generated by electroporating 1×10⁷ Jurkat T cells (Gene Pulser II, Bio-Rad) resuspended in 400 μl serum free medium with 100 μg of plasmid DNA. To select for the stable integration of expression plasmids, 2 mg/ml G418 (Gibco) was added to the culture medium 24 hours after transfection for 10 days. GFP-positive cells were further enriched by cell sorting using a MoFlo high-speed sorter (Dako).

### Microscopy.

HEK293T cells were grown overnight on glass coverslips coated with poly-L-lysine (Sigma-Aldrich). Upon harvesting, cells were rinsed with PBS before fixing with 10% phosphate buffered formalin and treated with Hoechst 33342 (Invitrogen) to stain the nuclei. Coverslips were mounted onto glass slides using ProLong Gold Antifade Reagent (Invitrogen). Epifluorescence microscopy was performed using a Nikon Eclipse E800 (Nikon Instruments). ImageJ was used for image analysis (NIH).

### Microarray Analysis.

Total RNA was isolated from stable Jurkat T cells expressing wild-type or mutant NLRC5, using TRIzol reagent (Invitrogen) according to the manufacturer's instruction. RNA aliquots were further cleaned up using the RNAeasy Mini kit (Qiagen) and subsequently analyzed on GeneChip Human Gene 1.0 ST Arrays (Affymetrix) at the Dana Farber Cancer Institute Microarray Core Facility. dChip was used to normalize array intensities to the array with the median overall intensity, and to calculate model-based expression values {Li, 2001 #42}. Sample comparisons and clustering analysis were also conducted using dChip (https://sites.google.com/site/dchipsoft/). Data were deposited in the Gene Expression Omnibus (GEO) database (accession no. GSE22064).

### Quantitative Real-Time PCR Analysis.

qRT-PCR analysis was performed as described and is detailed in SI Materials and Methods (42). Briefly, RNA samples were isolated using TRIzol reagent (Invitrogen) according to the manufacturer's instructions. The integrity of isolated RNA was verified by 1% agarose gel electrophoresis. First-strand cDNA was synthesized from 1 μg RNA using the qScript Flex cDNA synthesis kit (Quanta Biosciences), and RNA expression was quantified on the 7300 Real-Time PCR System (Applied Biosystems) using the PerfeCTa SYBR Green SuperMix with ROX (Quanta Biosciences). The following primers were used for amplification:

NLRC5 fwd
(SEQ ID NO: 29)
5′-CTGGCCAGTCTCACCGCACAA-3′
NLRC5 rev
(SEQ ID NO: 30)
5′-CCAGGGGACAGCCATCAAAATC-3′
HLA-A fwd
(SEQ ID NO: 31)
5′-AAAAGGAGGGAGTTACACTCAGG-3′
HLA-A rev
(SEQ ID NO: 32)
5′-GCTGTGAGGGACACATCAGAG-3′
HLA-B fwd
(SEQ ID NO: 33)
5′-CTACCCTGCGGAGATCA-3′
HLA-B rev
(SEQ ID NO: 34)
5′-ACAGCCAGGCCAGCAACA-3′
HLA-C fwd
(SEQ ID NO: 35)
5′-CACACCTCTCCTTTGTGACTTCAA-3′
HLA-C rev
(SEQ ID NO: 36)
5′-CCACCTCCTCACATTATGCTAACA-3′
TAP1 fwd
(SEQ ID NO: 37)
5′-AGGGCTGGCTGGCTGCTTTGA-3′
TAP1 rev
(SEQ ID NO: 38)
5′-ACGTGGCCCATGGTGTTGTTAT-3′
LMP2 fwd
(SEQ ID NO: 39)
5′-CGTTGTGATGGGTTCTGATTCC-3′
LMP2 rev
(SEQ ID NO: 40)
5′-GACAGCTTGTCAAACACTCGGTT-3′
β2M fwd
(SEQ ID NO: 41)
5′-TGCTGTCTCCATGTTTGATGTATCT-3′
β2M rev
(SEQ ID NO: 42)
5′-TCTCTGCTCCCCACCTCTAAGT-3′
DRA fwd
(SEQ ID NO: 43)
5′-GCCAACCTGGAAATCATGACA-3′
DRA rev
(SEQ ID NO: 44)
5′-AGGGCTGTTCGTGAGCACA-3′
CIITA fwd
(SEQ ID NO: 45)
5′-GGCTGGAATTTGGCAGCAC-3′
CIITA rev
(SEQ ID NO: 46)
5′-GCCCAACACAAGGATGTCTCT-3′
STAT1 fwd
(SEQ ID NO: 47)
5′-CCATCCTTTGGTACAACATGC-3′
STAT1 rev
(SEQ ID NO: 48)
5′-TGCACATGGTGGAGTCAGG-3′
GAPDH fwd
(SEQ ID NO: 49)
5′-GAAGGTGAAGGTCGGAGT-3′
GAPDH rev
(SEQ ID NO: 50)
5′-GAAGATGGTGATGGGATTTC-3′

The 7300 System SDS Software (Applied Biosystems) and Prism (GraphPad) were used for data analysis and graphing.

### Western Blotting.

Whole cell extracts were prepared using Cell Lysis Buffer (Cell Signaling) supplemented with 1 mM DTT and 1 mM PMSF, prior to extraction and centrifugation of whole cell lysates. Protein concentration was determined using the Bradford protein assay according to manufacturer's instructions (Bio-Rad). Cell extracts were subjected to SDS-polyacrylamide gel electrophoresis using 4-12% gradient gels (Invitrogen). Gels were transferred to polyvinylidene difluoride (PVDF) membranes (Millipore) for at least 3 hours at 80V. Membranes were blocked for 1 hour in 4% BSA in Tris-buffered saline—Tween (50 mM Tris, pH 7.6, 150 mM NaC1, 0.1% Tween 20). The following antibodies were used for protein detection: anti-GFP (JL-8, Clontech), anti-β2M (2M2, BioLegend), anti-LMP2 (LMP2-13, Biomol), anti-α-Tubulin (TU-02, Santa Cruz), and anti-β-Actin (1-19, Santa Cruz). Anti-TAP1 (R.RING4C) and anti-MHC class I heavy chain (3B10.7) are a kind gift from Dr. P. Cresswell (Yale University). The following horseradish peroxidase (HRP)-conjugated secondary antibodies were used: anti-mouse IgG and anti-rabbit IgG (GE Healthcare), anti-rat IgG2a (Alpha Diagnostics), and anti-goat IgG (Santa Cruz). Blots were developed using SuperSignal West Pico Chemiluminescent Substrate (Thermo Scientific), and imaged using the Molecular Imager ChemiDoc XRS+System (Bio-Rad). Image analysis was performed using Quantity One software (Bio-Rad).

### Chromatin Immunoprecipitation (ChIP) Assay.

Chromatin Immunoprecipitation of NLRC5 was performed as previously described (43). An anti-GFP antibody (JL-8, Clontech, 6 μg) was used to immunoprecipitate the corresponding GFP-fusion proteins from the stably transfected Jurkat T cell lines described above or from transiently transfected HEK293T cells. Purified DNA was analysed by quantitative real-time PCR using the following primers:

HLA-A fwd
(SEQ ID NO: 51)
5′-TCCGCAGTTTCTTTTCTCCC-3′
HLA-A rev
(SEQ ID NO: 52)
5′-GGAGAATCTGAGTCCCGGTGG-3′
HLA-B fwd
(SEQ ID NO: 53)
5′-TCTCAGGGTCTCAGGCTCCGAG-3′
HLA-B rev
(SEQ ID NO: 54)
5′-TGCGTGGGGACTTTAGAACTGG-3′
HLA-DRA fwd
(SEQ ID NO: 55)
5′-ATTTTTCTGATTGGCCAAAGAGTAATT-3′
HLA-DRA rev
(SEQ ID NO: 56)
5′-AAAAGAAAAGAGAATGTGGGGTGTAA-3′
GAPDH fwd
(SEQ ID NO: 57)
5′-TACTAGCGGTTTTACGGGCG-3′
GAPDH rev
(SEQ ID NO: 58)
5′-TCGAACAGGAGGAGCAGAGAGCGA-3′

### Phylogenetic Analysis.

The phylogenetic tree of selected members of the human NLR family was constructed using the highly conserved NBD sequences, obtained from the following NCBI reference sequences: NOD1 (Aa 196-368) NP_{—}006083.1, NOD2 (Aa 293-463) NP_{—}071445.1, NLRC3 (Aa 139-305) NP_{—}849172.2, NLRC5 (Aa 222-382) NP_{—}115582.3, NLRX1 (Aa 160-325) NP_{—}078894.2, CIITA (Aa 414-585) NP_{—}000237.2. ClustalW (EMBL-EBI) was used for sequence alignment and clustering.

Results

### NLRC5 Contains a Nuclear Localization Signal and Shuttles Between the Cytosol and the Nucleus.

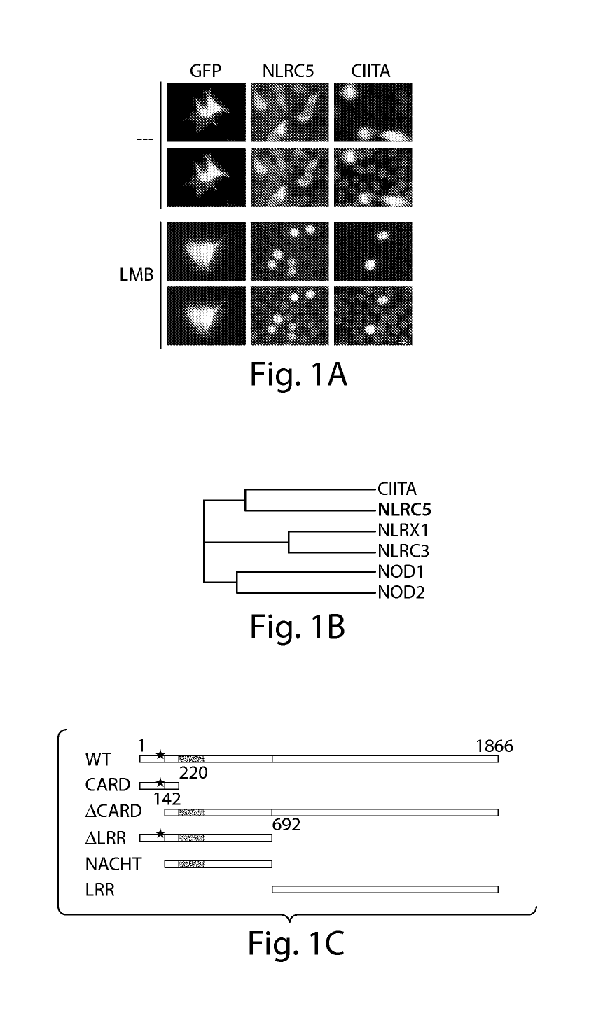

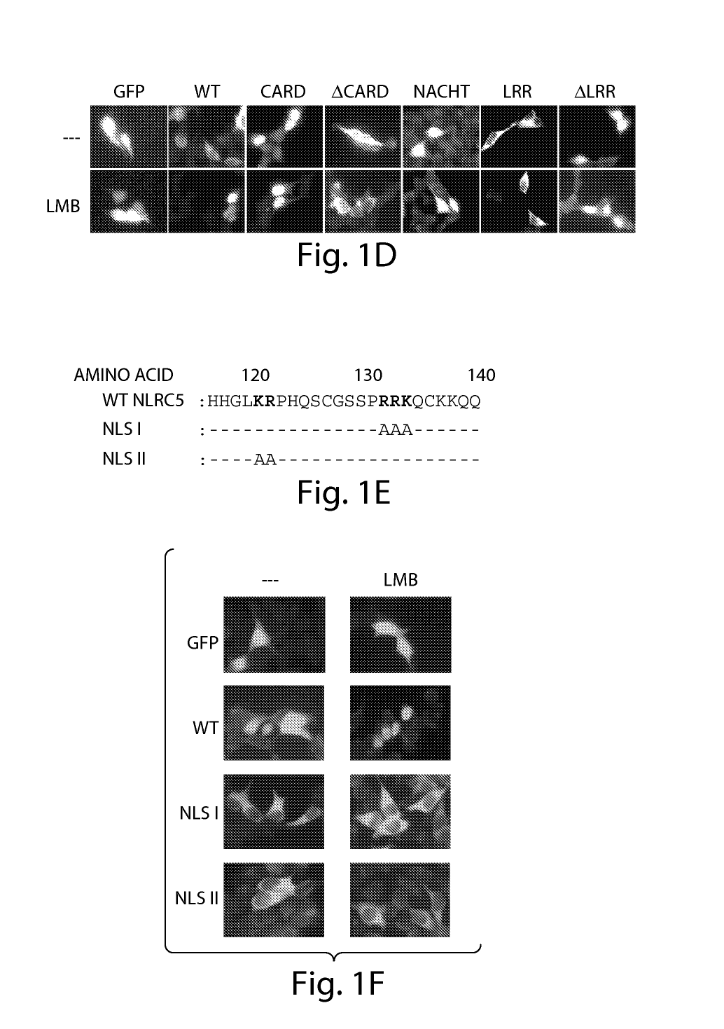

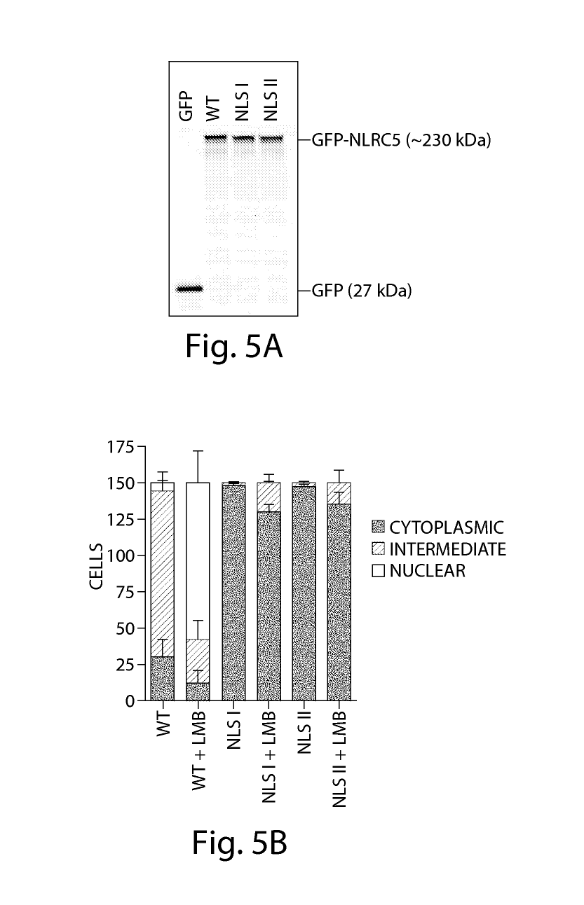

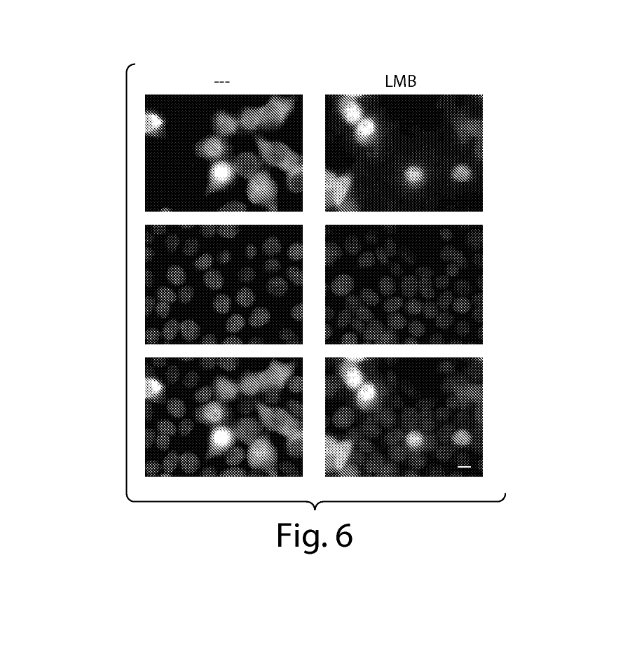

In order to study the function of NLRC5, its cellular distribution was investigated using a GFP-fusion protein. Surprisingly, NLRC5 was found not only in the cytosol, but also in the nucleus (FIG. 1A, upper panel). The stability of the fusion protein was checked by Western blot analysis, confirming that its nuclear localization was not due to a smaller, GFP-containing cleavage product (FIG. 5A). It has been demonstrated that CIITA, which also displays a heterogeneous steady-state distribution, shuttles between the nucleus and the cytosol as a result of NLS-mediated nuclear import and CRM1-dependent nuclear export (24-26). Similar to CIITA, which is a closely related member of the NLR protein family (FIG. 1B), NLRC5 could be trapped in the nucleus upon treatment with the CRM1 inhibitor leptomycin B (LMB) (FIG. 1A, lower panel and FIG. 5B). Quantification of the cellular distribution in a blind manner revealed that under steady state conditions, NLRC5 localized exclusively in the cytosol in approximately 15% of the cells. The majority of the cells showed an intermediate distribution (80%), and about 5% of the cells displayed an exclusively nuclear localization (FIG. 5B). LMB treatment resulted in nuclear localization of NLRC5 in more than 75% of the cells. Of note, it was observed that in cells highly expressing the protein, NLRC5 was predominantly localized to the cytosol, while NLRC5 was found more frequently in the nucleus in cells with lower expression levels, indicating that the nuclear localization of NLRC5 is not a result of overexpression (FIG. 1A upper panel). In addition to human NLRC5, similar results were obtained using the murine Nlrc5, which can also be trapped in the nucleus upon LMB treatment (FIG. 6).

Given the predicted size of the NLRC5 fusion protein (˜230 kDa), passive diffusion through the nuclear pore is not possible. Active transport, however, requires the presence of a nuclear localization signal (NLS) that is recognized by nuclear import receptors (27). In order to identify the NLS of NLRC5, deletion mutant analysis was performed. As depicted in FIG. 1C, the deletion mutants of NLRC5 were expressed as GFP fusion proteins. While all fusion constructs containing an intact N-terminal CARD (WT, CARD, ΔLRR) were found in the nucleus, deletion of the CARD (ΔCARD, LRR) resulted in a strictly cytosolic localization (FIG. 1D). Similar to free GFP, the NACHT domain fusion protein was found in both the nucleus and cytosol, presumably due to passive diffusion as a result of its smaller size. These results suggested that an NLS may be located in the N-terminal CARD. Indeed, sequence analysis of NLRC5 revealed a putative bipartite NLS at the transition between the CARD and the NBD (FIG. 1E) (25, 26). As predicted, mutation of the NLS abolished nuclear localization under steady state conditions, and treatment of the cells with LMB failed to trap the NLS mutants of NLRC5 in the nucleus (FIG. 1F). Taken together, the results demonstrate that, similar to the transcriptional co-regulator CIITA, NLRC5 shuttles between the cytosol and the nucleus and is thus likely to have a nuclear function.

### NLRC5 Transcriptionally Induces the Expression of MHC Class I and Functionally Related Genes.

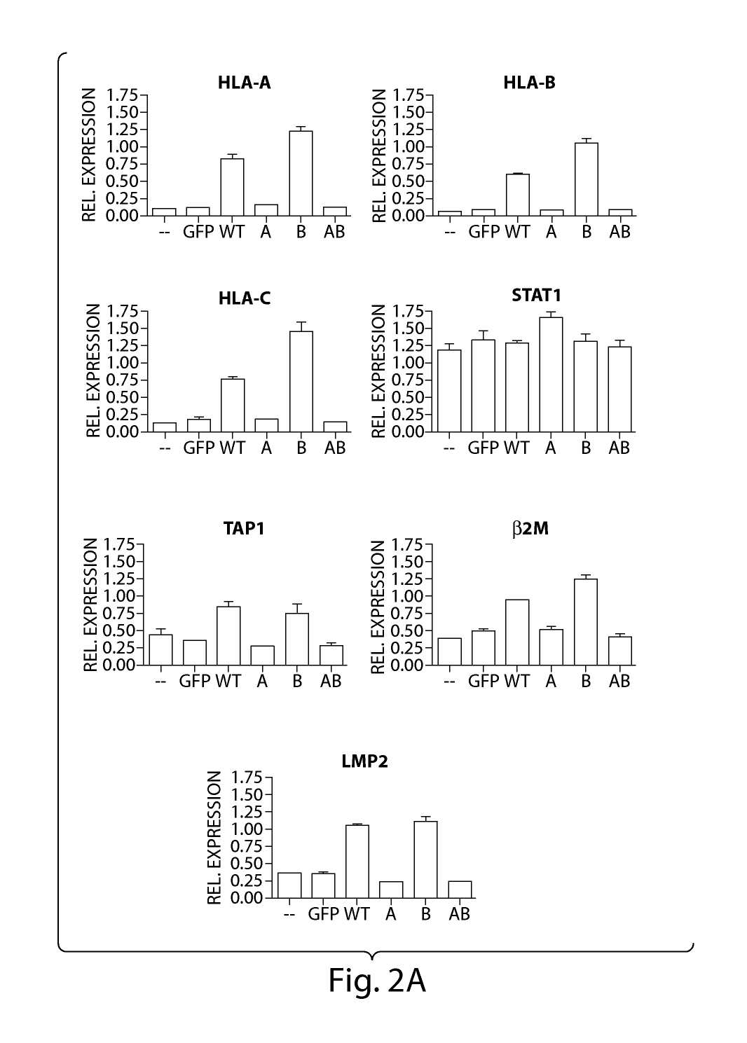

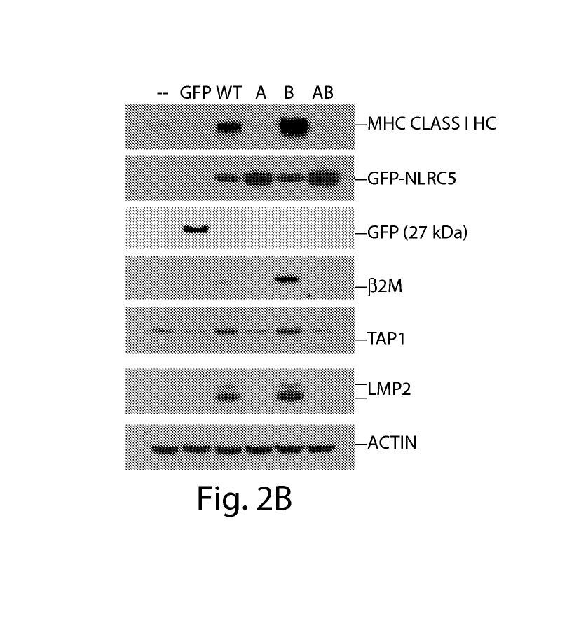

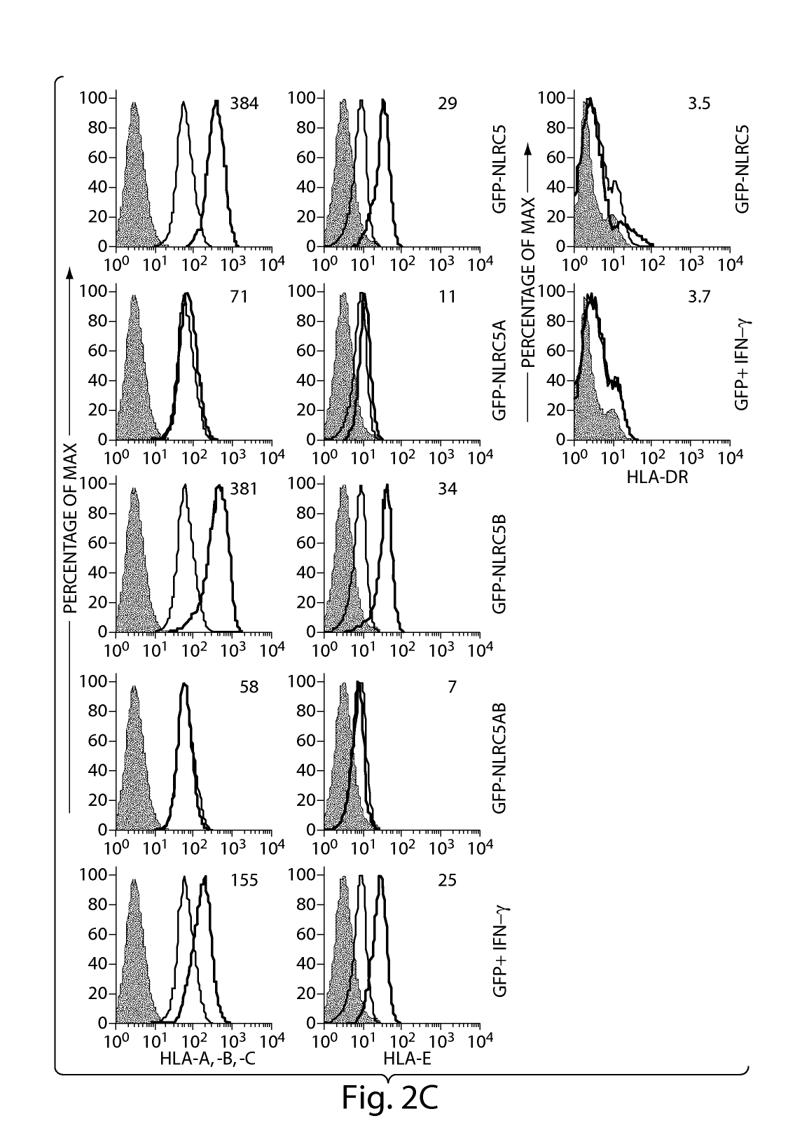

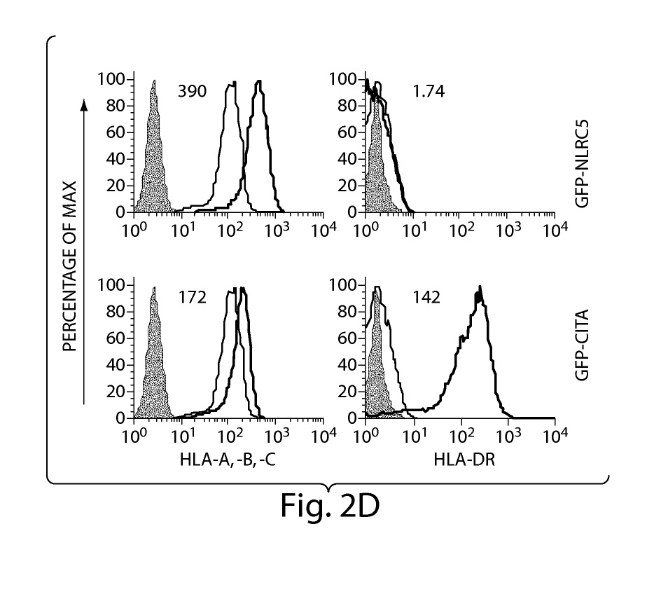

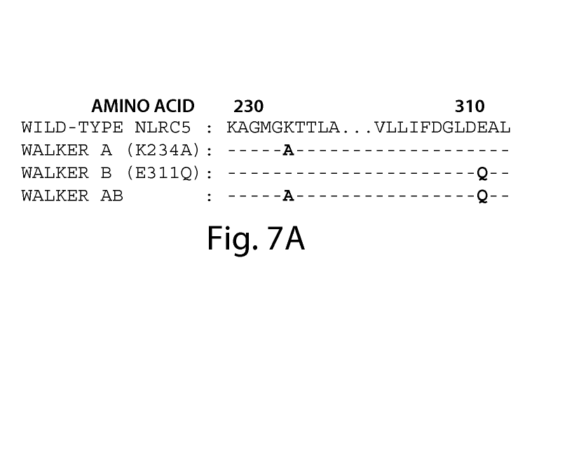

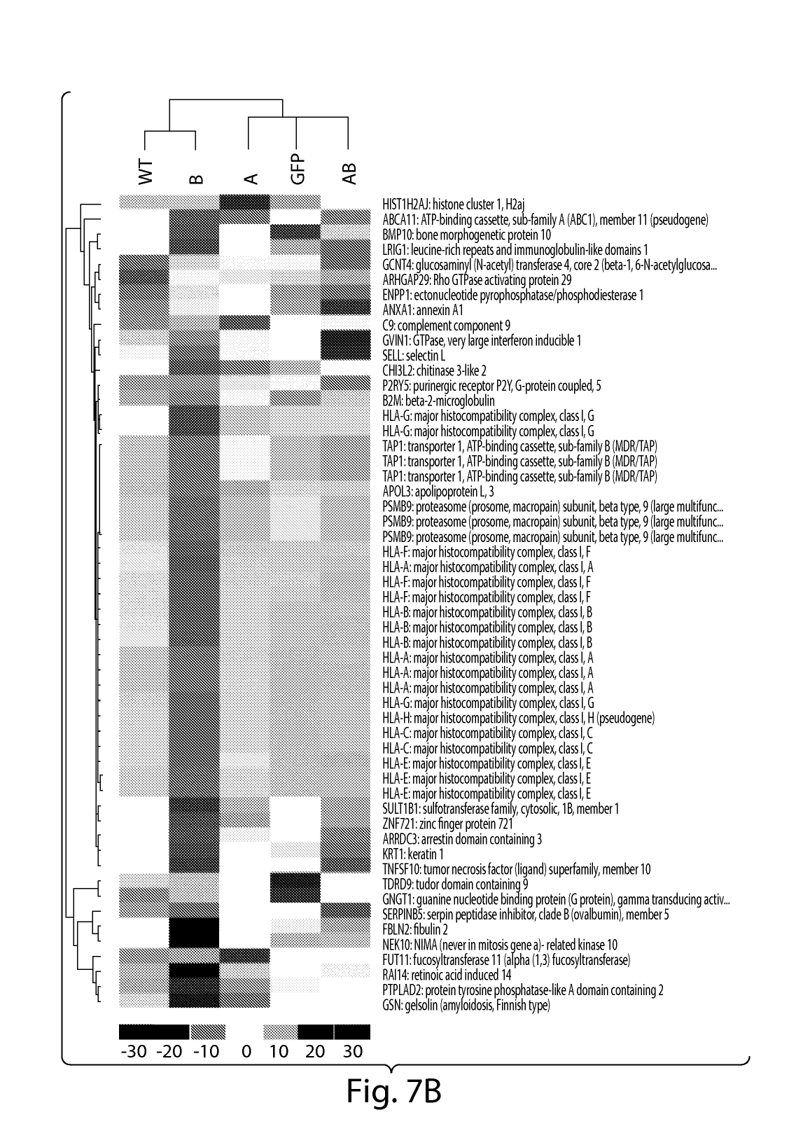

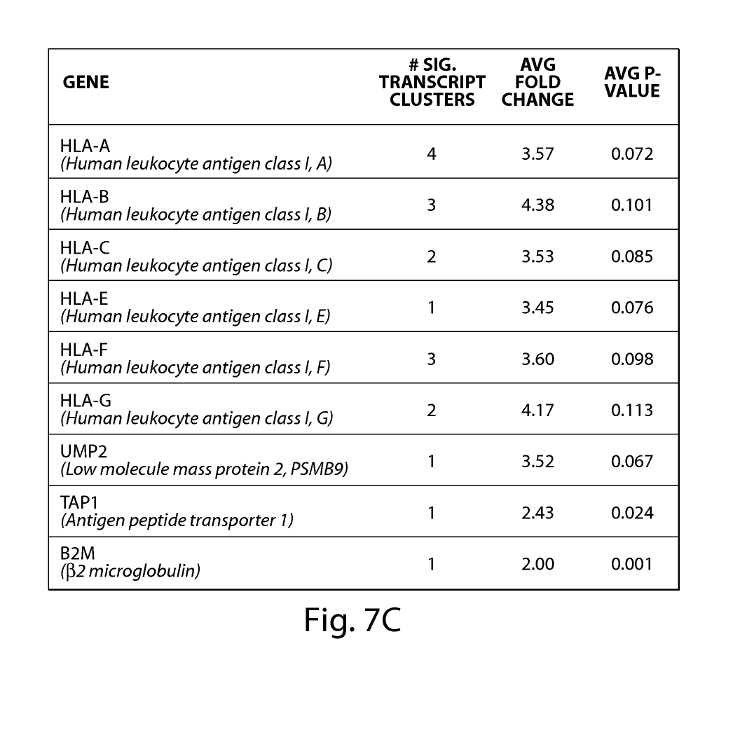

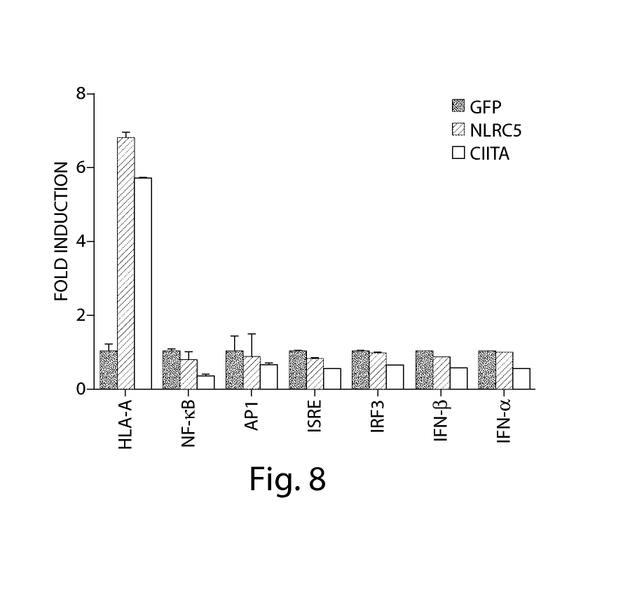

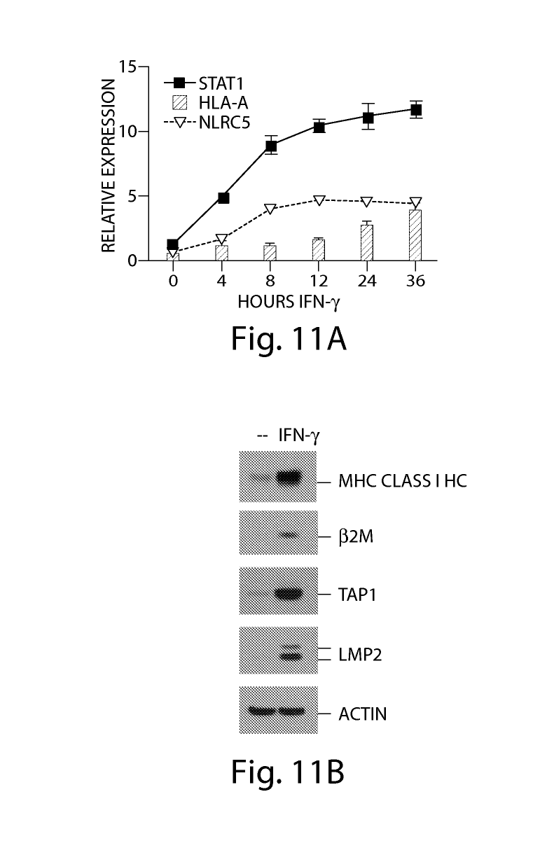

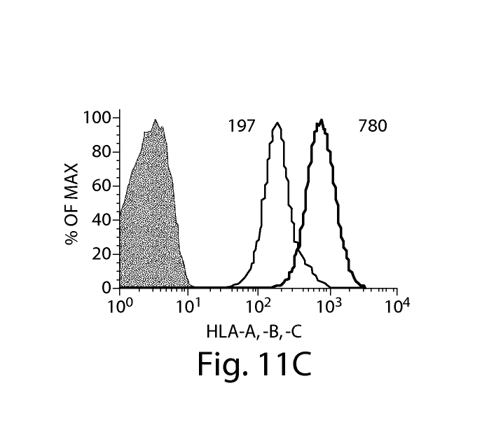

NLRC5 is also found in the nucleus and shares significant sequence similarity to the transcriptional co-regulator CIITA (FIGS. 1A and B). A gene array was performed to identify putative target genes of NLRC5. For this purpose, Jurkat T cell lines were generated that stably express either the wild-type or mutant forms of NLRC5 harboring mutations in the nucleotide binding domain (NBD): Walker A (deficient in nucleotide binding), Walker B (deficient in nucleotide hydrolysis), and the combined Walker AB, carrying both mutations (FIG. 7A) (28). Gene chip analysis using these mutant Jurkat T cells showed that a surprisingly limited number of genes were differentially regulated (FIG. 7). As predicted, clustering analysis grouped the active forms of NLRC5 (WT and Walker B) together, and they show a strikingly different pattern of gene expression compared to cells expressing either GFP alone, or the catalytically inactive forms of NLRC5 (Walker A and Walker AB). Amongst the genes most upregulated by the active forms of NLRC5 were the various members of the MHC class I (HLA-A, -B, -C, -E) family as well as other genes involved in class I antigen presentation and processing, such as 62M, LMP2 and TAP1 (FIGS. 7B and 7C). qRT-PCR and Western blot analysis confirmed elevated levels of the corresponding transcripts and proteins, respectively, in cells expressing the WT and Walker B mutant NLRC5, but not GFP alone, or the inactive forms of NLRC5 (Walker A and Walker AB) (FIGS. 2A and B). Furthermore, flow cytometry analysis using a pan HLA-A, -B, -C antibody, and an antibody specific for HLA-E, confirmed an increase in MHC class I surface expression in cells expressing NLRC5 WT or the Walker B mutant (FIG. 2C left). As previously shown, MHC class I and related genes are inducible by IFN-γ (FIG. 2C bottom and FIG. 11B) (5, 29). However, elevated levels of IFN-γ expression were not observed in our gene array analysis, and the expression level of STAT1, an IFN-γ-inducible gene, did not vary between the different cell lines (FIG. 2A). These findings, along with the observation that overexpression of NLRC5 does not activate NF-κB-, AP-1-, ISRE- or IRF3-dependent reporter genes, or the promoters of IFN-α and IFN-β (FIG. 8), rule out the role of these other pathways in NLRC5-mediated MHC class I induction. Instead, NLRC5 might directly regulate the expression of MHC class I genes.

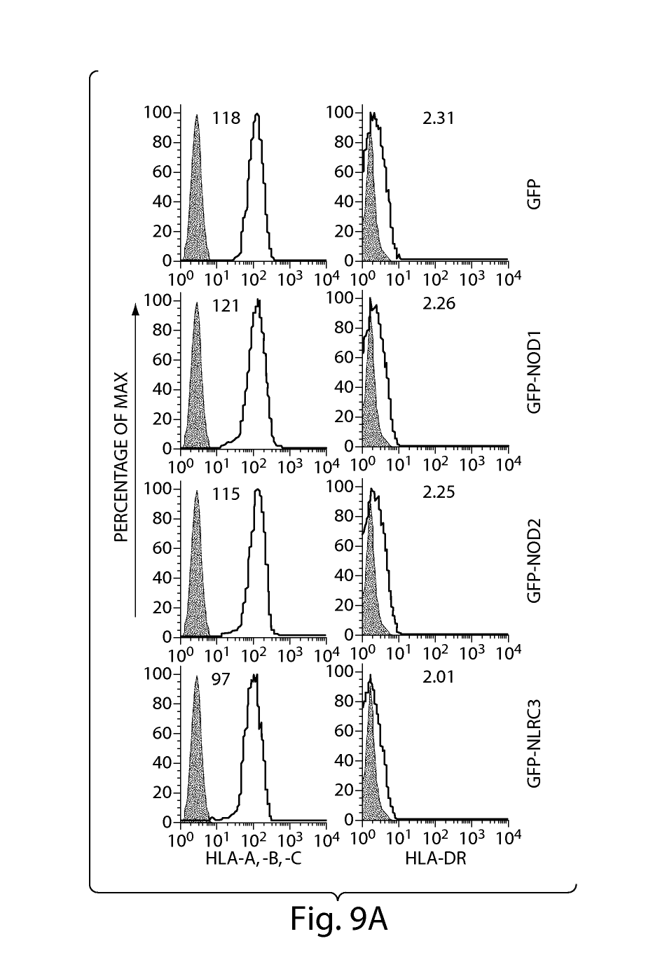

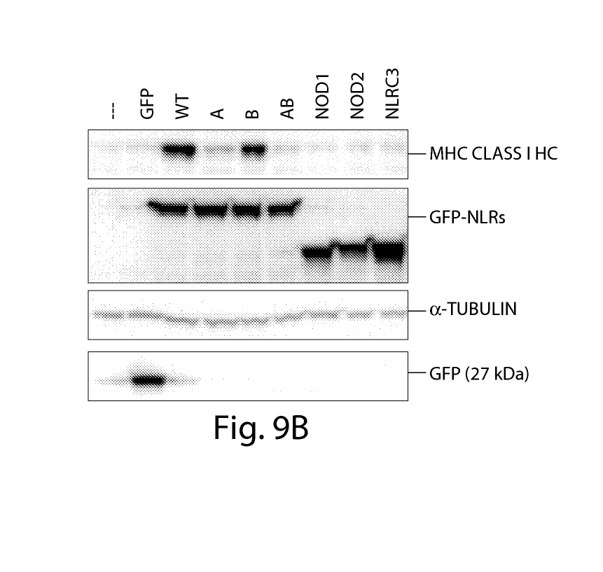

Since MHC class I is ubiquitously expressed in all nucleated cells, the inventors sought to determine whether the observed upregulation of MHC class I genes was limited to lymphoid cells, or could be extended to other cell types. As shown in FIG. 2D, transient expression of NLRC5 in an epithelial cell line (HEK293T cells) also increased MHC class I expression nearly fourfold. In comparison, expression of CIITA only moderately increased MHC class I expression but, in agreement with previous reports (6, 9), strongly induced the expression of MHC class II. Expression of the Walker A and B mutants in HEK293T cells (FIG. 9B) again demonstrated that nucleotide binding, but not nucleotide hydrolysis, was required for the activity of NLRC5 and the induction of MHC class I. Importantly, this transcriptional effect seems to be specific for the nuclear NLRs, since none of the cytosolic CARD-containing NLRs tested (NOD1, NOD2, NLRC3) increased the expression of MHC class I as demonstrated by flow cytometry and Western blot analysis (FIGS. 9A and 9B). In summary, these data indicate that NLRC5 induces the expression of MHC class I and related genes involved in MHC class I antigen presentation and thus can substitute for IFN-γ stimulation of cells.

NLRC5 Binds to MHC Class I Promoters and Induces their Expression.

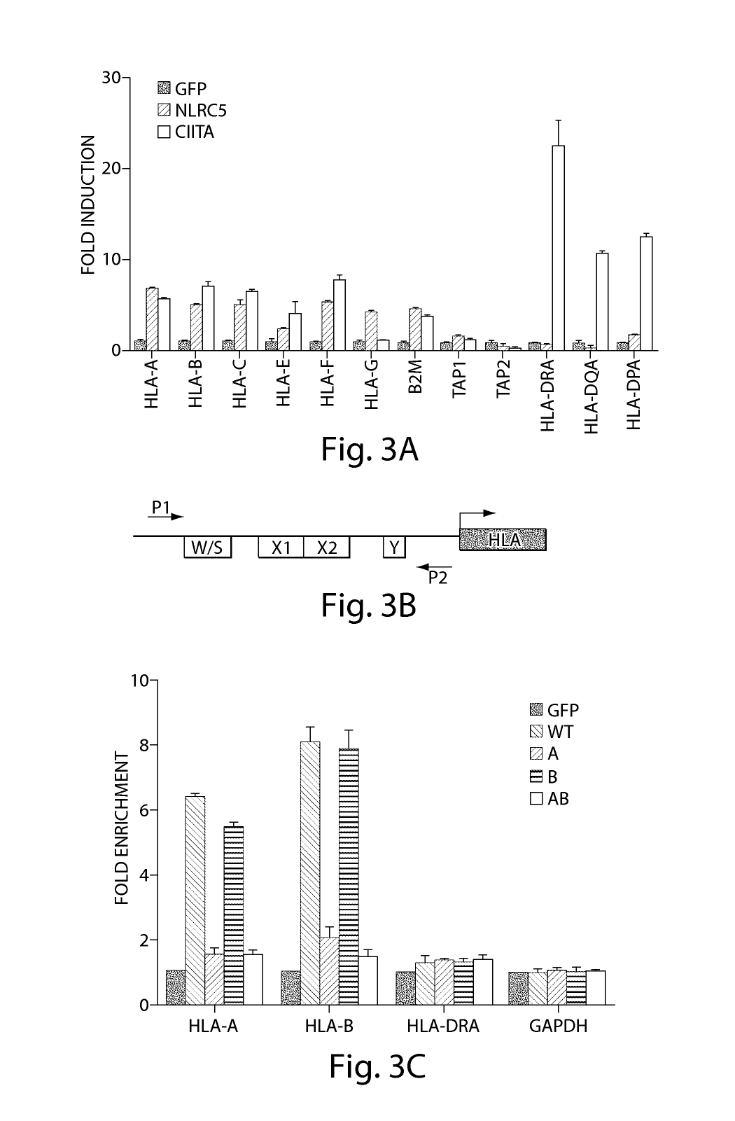

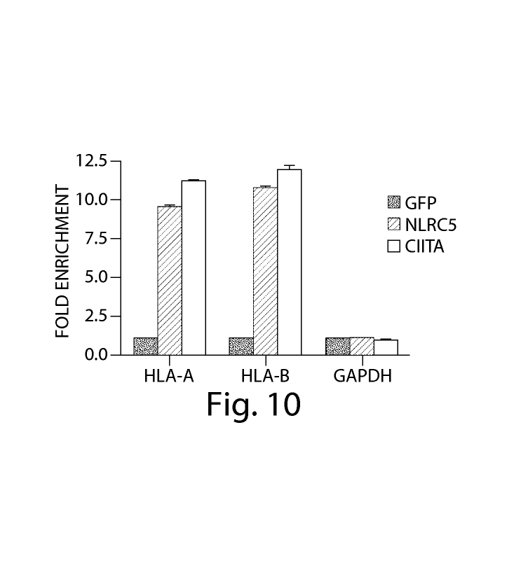

In order to investigate whether NLRC5 directly acts on the promoters of the MHC class I genes, luciferase-reporter gene assays were performed with the promoters of the corresponding genes. Transient expression of NLRC5 in HEK293T cells is sufficient to induce luciferase expression from the promoters of HLA-A, -B, -C, -F, -G, and β2M (FIG. 3A). Similar levels of induction on the same promoters were observed when CIITA was overexpressed, as has been reported previously (8, 29-31). Only a minor induction was observed on the promoter of TAP1, and NLRC5 failed to induce luciferase expression on the TAP2 promoter and any of the MHC class II reporter constructs analyzed (HLA-DRA, -DQA, -DPA). In contrast, CIITA transfection strongly activated the promoters of MHC class II genes. Next, the inventors examined if NLRC5 also physically associates with the MHC class I promoters using the stable Jurkat T cell lines described earlier in a chromatin immunoprecipitation (ChIP) assay. The corresponding wild-type and mutant NLRC5 proteins were immunoprecipitated, and the associated DNA fragments were quantified by qPCR, using gene specific primers covering the immediate upstream region of the HLA genes (FIG. 3B). As seen in FIG. 3C, a 6- to 8-fold enrichment in promoter occupancy was observed for NLRC5 WT and the Walker B mutant on the promoters of HLA-A, and HLA-B when compared to the cell line expressing GFP only. In agreement with the data obtained from the gene expression analyses, no promoter binding was detected for the inactive forms of NLRC5 (Walker A and Walker AB), as well as on the promoter of the MHC class II (HLA-DRA), and an unrelated gene (GAPDH). Furthermore, ChIP analysis in non-hematopoietic cells using transiently transfected HEK293T cells revealed that NLRC5 can associate with HLA-A and -B promoters to a similar extent as CIITA (FIG. 10), which has previously been reported to bind to MHC class I promoters (10). Taken together, the luciferase assay and the ChIP experiment demonstrate that NLRC5 not only associates with the promoters of the MHC class I genes with remarkable specificity, but also has the capacity to transactivate their expression.

### NLRC5 is Rapidly Induced by IFN-γ and is Required for IFN-γ-Induced Expression of MHC Class I.

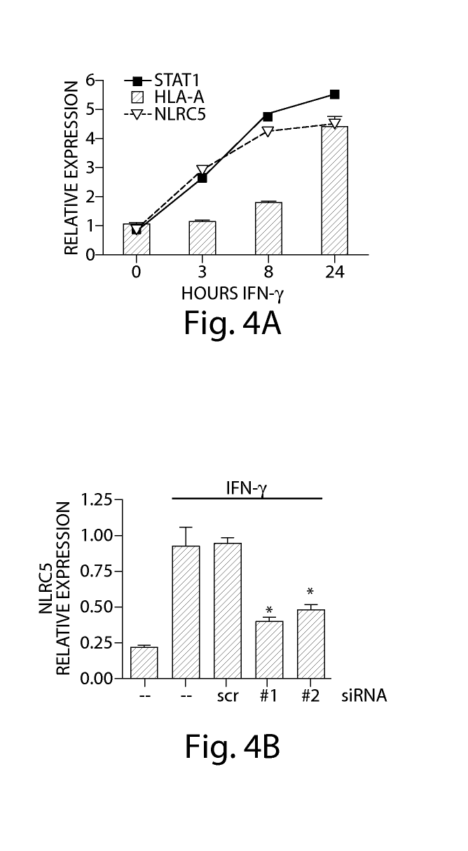

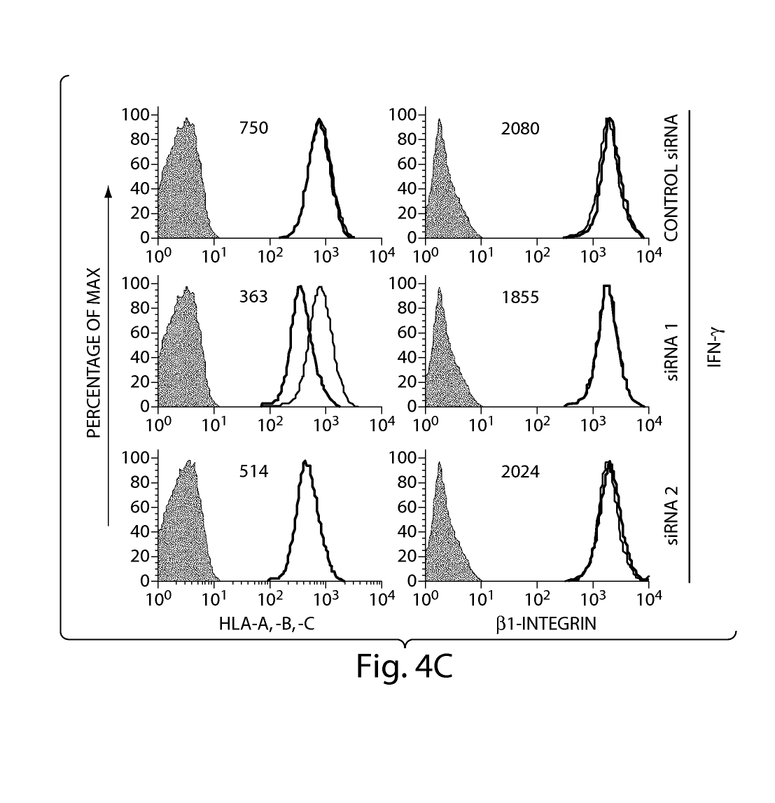

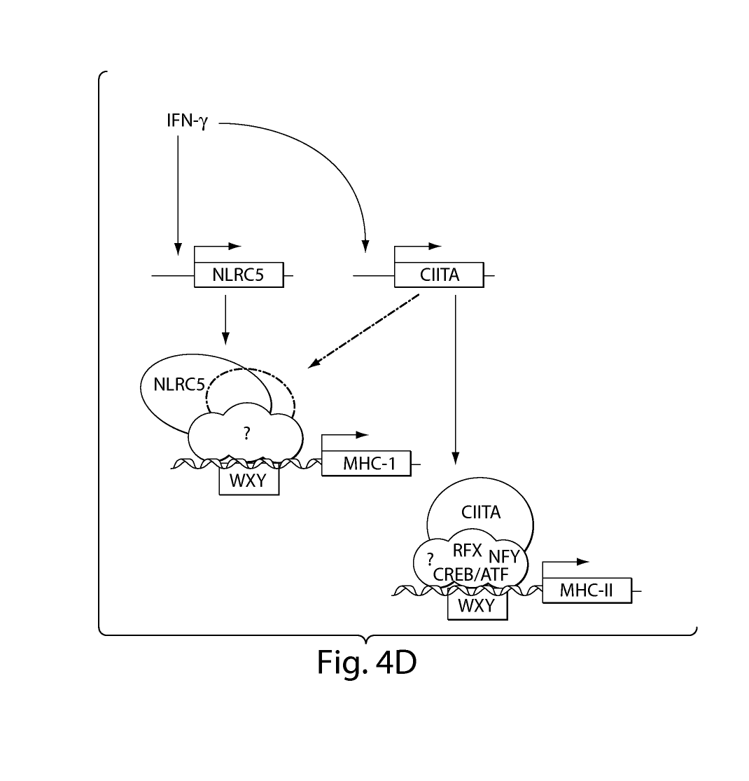

It has been shown that rapid induction of CIITA mediates the upregulation of MHC class II upon IFN-γ stimulation (15, 32). As NLRC5 is also an IFN-γ-inducible gene (33), the possibility that NLRC5 may mediate the IFNγ-induced transcription of MHC class I genes was explored. First, the expression kinetics of NLRC5 and a MHC class I gene upon IFN-γ treatment was compared. HLA-A transcript levels reach a maximum only 12-24 hrs after IFN-γ stimulation in HeLa cells but, similar to the IFN-γ-response gene STAT1, NLRC5 is induced early after IFN-γ treatment (FIG. 4A), which is also a characteristic of CIITA induction by IFN-γ (15, 32). Similar kinetics of NLRC5 and HLA-A expression were observed in Jurkat T cells (FIG. 11A).

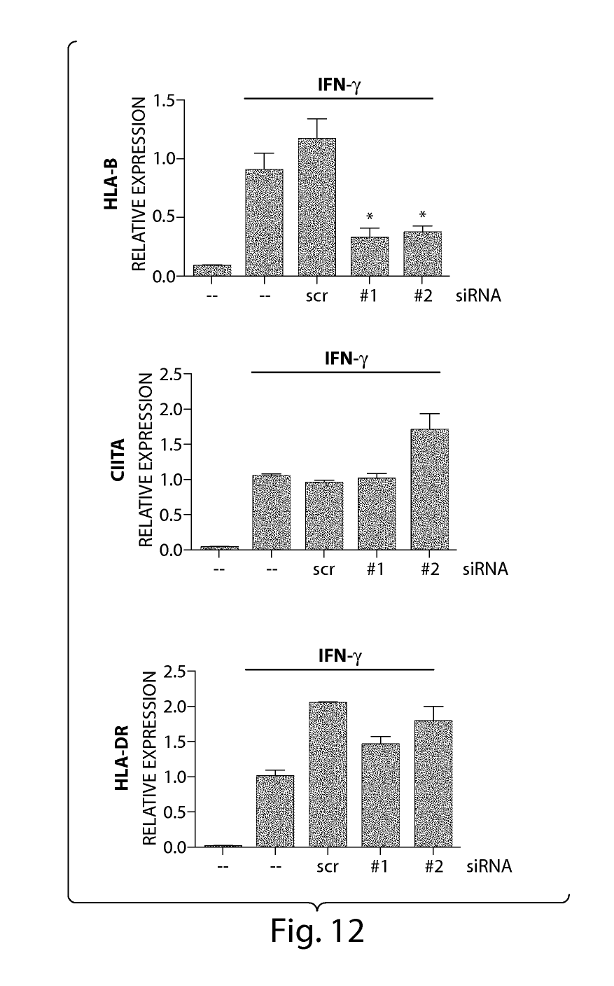

Next, the effect of NLRC5 depletion by RNA interference on the expression of MHC class I after IFN-γ stimulation was analyzed. We had observed earlier that surface expression of MHC class I is readily induced upon IFN-γ stimulation (FIG. 11C), and in agreement with our hypothesis, transfection of HeLa cells with two different NLRC5-specific siRNAs, but not a scrambled control siRNA, significantly reduced the IFN-γ-induced upregulation of MHC class I (FIG. 4C left panel and FIG. 12). In contrast, an unrelated, but IFN-γ-inducible, surface receptor, β1-integrin, was not affected by the depletion of NLRC5 (FIG. 4C right panel). Similarly, the IFN-γ-induced expression of CIITA and HLA-DR was not affected by the depletion of NLRC5 (FIG. 12), strongly suggesting that NLRC5 is required for the efficient induction of MHC class I observed upon IFN-γ stimulation.

Since the complementation cloning of CIITA from MHC class II deficient patients in 1993, CIITA has been often referred to as a “master regulator” of MHC class II expression as CIITA is required for both constitutive and IFN-γ-inducible transcription of MHC class II genes (15, 20, 32). However, the contribution of CIITA to MHC class I expression is less clear. In this study, NLRC5 was identified as a novel regulator of MHC class I genes in addition to CIITA. NLRC5 and CIITA share important characteristics in their structure and function. First, as related members of the NLR family (FIG. 1B), both have the same tripartite architecture, although expression of the CARD-containing isoform of CIITA is limited to dendritic cells (34). Interestingly, both proteins require an active NBD for their function. It has been shown that the NTP binding motif in CIITA is essential for transactivation of MHC class II genes (28, 35, 36). Similarly, the Walker A mutation, which prevents NTP binding, but not the Walker B mutation, which abolishes NTP hydrolysis, resulted in a loss of NLRC5 function (FIG. 2). Second, both proteins can localize to the nucleus. CIITA carries three NLSs, including an N-terminal NLS, which is found at a similar position to that required for NLRC5 nuclear translocation (FIGS. 1E and F) (24-26). In addition, multiple nuclear export signals (NES) are predicted in the C-terminal LRRs of CIITA, and the deletion mutant analysis suggests that the C-terminal LRRs of NLRC5 are also involved in the regulation of nuclear export, although the exact position of the NES needs to be mapped (26). Recently it was shown that cytosolic NLRC5 negatively regulates the NF-κB and type I IFN signaling pathway by direct binding to IKK and RIG-I (37). The findings described herein do not rule out a function of NLRC5 in the cytoplasm, but rather demonstrate its novel role in the nucleus as a transcriptional regulator of MHC class I genes. Third, despite the lack of a DNA-binding domain, both NLRC5 and CIITA can associate with and transactivate MHC class I promoters (FIGS. 3A and C, and FIG. 10) (10, 17, 29). CIITA is known to associate with a set of transcription factor complexes, or ‘MHC enhanceosome’, on the WXY motif of the MHC class I and class II gene promoters. The results of the ChIP and reporter gene assays indicate that NLRC5 may use a similar platform to activate MHC class I gene promoters. Finally, both NLRC5 and CIITA are highly inducible upon IFN-γ stimulation (FIG. 4A) (15, 32, 33), and binding sites for STAT1, which is activated upon IFN-γ stimulation, have been mapped in the promoters of both genes (33, 38-40). This suggests that both proteins are involved in mediating IFN-γ-induced changes in gene expression. In particular, CIITA and NLRC5 appear to orchestrate the concerted expression of sets of functionally related genes critical for antigen presentation. CIITA, in addition to the classical MHC class II genes, induces the invariant chain Ii, and the non-classical MHC class II genes HLA-DM, HLA-DO, which play accessory roles in MHC class II antigen presentation (16). NLRC5, beyond the induction of MHC class I genes, upregulates β2M, TAP1 and LMP2, which are essential for antigen presentation by the MHC class I pathway (FIG. 2A).

However, in spite of these similarities and overlapping functions, there are also noticeable differences between NLRC5 and CIITA. A unique feature of NLRC5 is its striking specificity for the induction of genes involved in the MHC class I pathway, as opposed to CIITA which can induce both MHC class I and class II genes. The expression of NLRC5 in epithelial and lymphoid cells was found to be sufficient to induce MHC class I but not MHC class II genes, despite their similar promoter architecture (FIGS. 2C and D). Furthermore, the findings described here also suggest that NLRC5 is exclusively associated with the promoters of MHC class I (FIG. 3C), and NLRC5 transactivated promoters of MHC class I and related genes but not those of MHC class II genes (FIG. 3A). A possible explanation for this specificity could lie in the structural differences between the two proteins. NLRC5, unlike CIITA, lacks N-terminal acidic and proline/serine/threonine-rich domains, which are required for MHC class II promoter activation (41). NLRC5 will thus require additional co-factors to interact with and activate the enhanceosome found on the MHC class I promoters.

Given its specificity for MHC class I induction, it is also possible that NLRC5 plays a dominant role in the regulation of MHC class I gene expression. This view is supported by the results of our knockdown analyses, which clearly show that the IFN-γ-induced upregulation of CIITA cannot compensate for the reduction in MHC class I expression observed upon NLRC5 depletion (FIG. 4C and FIG. 12). Furthermore, no reduction in MHC class I expression has been observed in CIITA-deficient mice (21-23). Taken together, the findings described herein demonstrate that NLRC5 is necessary and sufficient for the induction of MHC class I expression. NLRC5 may thus act as a counterpart to CIITA in its function as an “MHC class I transactivator” or “CITA”. Future analyses of the in vivo function of NLRC5 are required to reveal if these two molecules play redundant or more exclusive roles in MHC class I-dependent immune responses.

Without intending to be bound by theory, the following model of NLRC5 function in the expression of MHC class I genes is proposed: Upon IFN-γ stimulation, activated STAT1 acts on the promoters of NLRC5 and CIITA and rapidly induces these genes (FIG. 4D). Subsequently, CIITA may activate the promoters of both MHC class I and class II genes by associating with the MHC enhanceosome, which includes the RFX, CREB/ATF and NF-Y protein complexes on the conserved WXY module in the MHC promoters (FIG. 4D). NLRC5 may also associate with a similar enhanceosome on the MHC class I promoter, consisting of the same or similar components as those described for the CIITA enhanceosome. However, unlike the CIITA enhanceosome, the NLRC5 enhanceosome is specific to promoters of MHC class I and of related genes (FIG. 4D).

### REFERENCES

1. Pamer E, Cresswell P (1998) Mechanisms of MHC class I—restricted antigen processing. Annu Rev Immunol 16:323-58.
2. Braud V M, Allan D S, McMichael A J (1999) Functions of nonclassical MHC and non-MHC-encoded class I molecules. Curr Opin Immunol 11:100-8.
3. Le Bouteiller P, Solier C (2001) Is antigen presentation the primary function of HLA-G? Microbes Infect 3:323-32.
4. Shastri N, et al. (2005) All the peptides that fit: the beginning, the middle, and the end of the MHC class I antigen-processing pathway. Immunol Rev 207:31-41.
5. Peaper D R, Cresswell P (2008) Regulation of MHC class I assembly and peptide binding. Annu Rev Cell Dev Biol 24:343-68.
6. Reith W, Mach B (2001) The bare lymphocyte syndrome and the regulation of MHC expression. Annu Rev Immunol 19:331-73.
7. van den Elsen P J, Gobin S J, van Eggermond M C, Peijnenburg A (1998) Regulation of MHC class I and II gene transcription: differences and similarities. Immunogenetics 48:208-21.
8. van den Elsen P J, Peijnenburg A, van Eggermond M C, Gobin S J (1998) Shared regulatory elements in the promoters of MHC class I and class II genes. Immunol Today 19:308-12.
9. Boss J M, Jensen P E (2003) Transcriptional regulation of the MHC class II antigen presentation pathway. Curr Opin Immunol 15:105-11.
10. Masternak K, et al. (2000) CIITA is a transcriptional coactivator that is recruited to MHC class II promoters by multiple synergistic interactions with an enhanceosome complex. Genes Dev 14:1156-66.
11. Wilmanski J M, Petnicki-Ocwieja T, Kobayashi K S (2008) NLR proteins: integral members of innate immunity and mediators of inflammatory diseases. J Leukoc Biol 83:13-30.
12. Ting J P, Willingham S B, Bergstralh D T (2008) NLRs at the intersection of cell death and immunity. Nat Rev Immunol 8:372-9.
13. Beresford G W, Boss J M (2001) CIITA coordinates multiple histone acetylation modifications at the HLA-DRA promoter. Nat Immunol 2:652-7.
14. DeSandro A, Nagarajan U M, Boss J M (1999) The bare lymphocyte syndrome: molecular clues to the transcriptional regulation of major histocompatibility complex class II genes. Am J Hum Genet 65:279-86.
15. Steimle V, et al. (1994) Regulation of MHC class II expression by interferon-gamma mediated by the transactivator gene CIITA. Science 265:106-9.
16. LeibundGut-Landmann S, et al. (2004) Mini-review: Specificity and expression of CIITA, the master regulator of MHC class II genes. Eur J Immunol 34:1513-25.
17. Martin B K, et al. (1997) Induction of MHC class I expression by the MHC class II transactivator CIITA. Immunity 6:591-600.
18. Ting J P, Trowsdale J (2002) Genetic control of MHC class II expression. Cell 109 Suppl:S21-33.
19. Benichou B, Strominger J L (1991) Class II-antigen-negative patient and mutant B-cell lines represent at least three, and probably four, distinct genetic defects defined by complementation analysis. Proc Natl Acad Sci USA 88:4285-8.
20. Steimle V, Otten L A, Zufferey M, Mach B (1993) Complementation cloning of an MHC class II transactivator mutated in hereditary MHC class II deficiency (or bare lymphocyte syndrome). Cell 75:135-46.
21. Chang C H, et al. (1996) Mice lacking the MHC class II transactivator (CIITA) show tissue-specific impairment of MHC class II expression. Immunity 4:167-78.
22. Itoh-Lindstrom Y, et al. (1999) Reduced IL-4-, lipopolysaccharide-, and IFN-gamma-induced MHC class II expression in mice lacking class II transactivator due to targeted deletion of the GTP-binding domain. J Immunol 163:2425-31.
23. Williams G S, et al. (1998) Mice lacking the transcription factor CIITA—a second look. Int Immunol 10:1957-67.
24. Cressman D E, Chin K C, Taxman D J, Ting J P (1999) A defect in the nuclear translocation of CIITA causes a form of type II bare lymphocyte syndrome. Immunity 10:163-71.
25. Spilianakis C, Papamatheakis J, Kretsovali A (2000) Acetylation by PCAF enhances CIITA nuclear accumulation and transactivation of major histocompatibility complex class II genes. Mol Cell Biol 20:8489-98.
26. Cressman D E, et al. (2001) Mechanisms of nuclear import and export that control the subcellular localization of class II transactivator. J Immunol 167:3626-34.
27. Lange A, et al. (2007) Classical nuclear localization signals: definition, function, and interaction with importin alpha. J Biol Chem 282:5101-5.
28. Hanson P I, Whiteheart S W (2005) AAA+ proteins: have engine, will work. Nat Rev Mol Cell Biol 6:519-29.
29. Gobin S J, Peijnenburg A, Keijsers V, van den Elsen P J (1997) Site alpha is crucial for two routes of IFN gamma-induced MHC class I transactivation: the ISRE-mediated route and a novel pathway involving CIITA. Immunity 6:601-11.
30. Girdlestone J (2000) Synergistic induction of HLA class I expression by RelA and CIITA. Blood 95:3804-8.
31. Gobin S J, et al. (2001) The MHC-specific enhanceosome and its role in MHC class I and beta(2)-microglobulin gene transactivation. J Immunol 167:5175-84.
32. Chang C H, Fontes J D, Peterlin M, Flavell R A (1994) Class II transactivator (CIITA) is sufficient for the inducible expression of major histocompatibility complex class II genes. J Exp Med 180:1367-74.
33. Kuenzel S, et al. The nucleotide-binding oligomerization domain-like receptor NLRC5 is involved in IFN-dependent antiviral immune responses. J Immunol 184:1990-2000.
34. Nickerson K, et al. (2001) Dendritic cell-specific MHC class II transactivator contains a caspase recruitment domain that confers potent transactivation activity. J Biol Chem 276:19089-93.
35. Wright K L, et al. (1998) CIITA stimulation of transcription factor binding to major histocompatibility complex class II and associated promoters in vivo. Proc Natl Acad Sci USA 95:6267-72.
36. Bewry N N, Bolick S C, Wright K L, Harton J A (2007) GTP-dependent recruitment of CIITA to the class II major histocompatibility complex promoter. J Biol Chem 282:26178-84.
37. Cui J, et al. NLRC5 negatively regulates the NF-kappaB and type I interferon signaling pathways. Cell 141:483-96.
38. Muhlethaler-Mottet A, Di Berardino W, Otten L A, Mach B (1998) Activation of the MHC class II transactivator CIITA by interferon-gamma requires cooperative interaction between Stat1 and USF-1. Immunity 8:157-66.
39. Piskurich J F, et al. (1998) Identification of distinct regions of 5′ flanking DNA that mediate constitutive, IFN-gamma, STAT1, and TGF-beta-regulated expression of the class II transactivator gene. J Immunol 160:233-40.
40. Piskurich J F, Linhoff M W, Wang Y, Ting J P (1999) Two distinct gamma interferon-inducible promoters of the major histocompatibility complex class II transactivator gene are differentially regulated by STAT1, interferon regulatory factor 1, and transforming growth factor beta. Mol Cell Biol 19:431-40.
41. Chin K C, Li G G, Ting J P (1997) Importance of acidic, proline/serine/threonine-rich, and GTP-binding regions in the major histocompatibility complex class II transactivator: generation of transdominant-negative mutants. Proc Natl Acad Sci USA 94:2501-6.
42. Petnicki-Ocwieja T, et al. (2009) Nod2 is required for the regulation of commensal microbiota in the intestine. Proc Natl Acad Sci USA 106:15813-8.
43. Iliopoulos D, Hirsch H A, Struhl K (2009) An epigenetic switch involving NF-kappaB, Lin28, Let-7 MicroRNA, and IL6 links inflammation to cell transformation. Cell 139:693-706.

This invention is not limited in its application to the details of construction and the arrangement of components set forth in the above description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

1. A method of modulating MHC class I gene expression by modulating NLRC5 expression and/or NLRC5 activity in a subject in need thereof, the method comprising:
administering to the subject a compound that modulates NLRC5 expression and/or NLRC5 activity in an amount effective to modulate MHC class I gene expression.

2. The method of claim 1, wherein the compound increases NLRC5 expression and/or NLRC5 activity, whereby MHC class I gene expression is increased.

3. The method of claim 1, wherein the compound decreases NLRC5 expression and/or NLRC5 activity, whereby MHC class I gene expression is decreased.

4. The method of claim 1, wherein the compound is selected from the group consisting of siRNA, a synthetic organic chemical, a peptide, a natural fermentation product, and a substance extracted from a microorganism, plant, animal or cell culture medium.

5. The method of claim 2, further comprising administering to the subject a compound that increases CIITA expression in an amount effective to increase MHC class I and MHC class II gene expression.

6. The method of claim 3, further comprising administering to the subject a compound that decreases CIITA expression in an amount effective to decrease MHC class I and MHC class II gene expression.

7. A method of reducing viral infection by increasing NLRC5 expression and/or NLRC5 activity in a subject in need thereof, the method comprising:
administering to the subject a compound that increases NLRC5 expression and/or NLRC5 activity in an amount effective to increase MHC class I gene expression and reduce the viral infection in the subject.

8. The method of claim 7, wherein the compound is selected from the group consisting of siRNA, a synthetic organic chemical, a peptide, a natural fermentation product, and a substance extracted from a microorganism, plant, animal or cell culture medium.

9. The method of claim 7, further comprising administering to the subject a compound that increases CIITA expression in an amount effective to increase MHC class I gene expression, and reduce the viral infection in the subject.

10. A method of inhibiting cancer by increasing NLRC5 expression and/or NLRC5 activity in a subject in need thereof, the method comprising:
administering to the subject a compound that increases NLRC5 expression and/or NLRC5 activity in an amount effective to increase MHC class I gene expression and inhibit cancer in the subject.

11. The method of claim 10, wherein the compound is selected from the group consisting of siRNA, a synthetic organic chemical, a peptide, a natural fermentation product, and a substance extracted from a microorganism, plant, animal or cell culture medium.

12. The method of claim 10, further comprising administering to the subject a compound that increases CIITA expression in an amount effective to increase MHC class I and MHC class II gene expression, and inhibit cancer in the subject.

13. A method of inhibiting tissue or organ rejection by decreasing NLRC5 expression in a subject in need thereof, the method comprising:
administering to the subject a compound that decreases NLRC5 expression and/or NLRC5 activity in an amount effective to decrease MHC class I gene expression and inhibit tissue or organ rejection in the subject.

14. The method of claim 13, wherein the compound is selected from the group consisting of siRNA, a synthetic organic chemical, a peptide, a natural fermentation product, and a substance extracted from a microorganism, plant, animal or cell culture medium.

15. The method of claim 13, further comprising administering to the subject a compound that decreases CIITA expression in an amount effective to decrease MHC class I and MHC class II gene expression and inhibit tissue or organ rejection in the subject.

16. A method of identifying a compound that increases NLRC5 expression and/or NLRC5 activity, the method comprising:
(a) contacting a test cell with a test compound, wherein the cell comprises a NLRC5 nucleic acid; and
(b) comparing the level of expression and/or activity of NLRC5 in the test cell to the level of expression and/or activity of NLRC5 in a cell, referred to as a control cell, that has not been contacted with the test compound,
wherein if the level of expression and/or activity of NLRC5 in the test cell is greater than the level of expression and/or activity in the control cell, the test compound is a compound that increases NlRC5 expression and/or NLRC5 activity.

17. The method of claim 16, further comprising comparing the level of expression of MHC class I genes in the test cell to the level of expression in the control cell, wherein if the level of expression of MHC class I genes in the test cell is greater than the level of expression in the control cell, the test compound is a compound that increases MHC class I gene expression.

18. A method of identifying a compound that decreases NLRC5 expression and/or NLRC5 activity, the method comprising:
(a) contacting a test cell with a test compound, wherein the cell comprises a NLRC5 nucleic acid; and
(b) comparing the level of expression and/or activity of NLRC5 in the test cell compared to a control cell that has not been contacted with the test compound;
wherein if the level of expression and/or activity of NLRC5 in the test cell is less than the level of expression and/or activity in the control cell, the test compound is a compound that decreases NLRC5 expression and/or NLRC5 activity.

19. The method of claim 18, further comprising comparing the level of expression of MHC class I genes in the test cell to the level of expression in the control cell, wherein if the level of expression of MHC class I genes in the test cell is greater than the level of expression in the control cell, the test compound is a compound that increases MHC class I gene expression.

20. A pharmaceutical composition comprising an antibody that binds NLRC5 and a pharmaceutically acceptable carrier.

21. The pharmaceutical composition of claim 20, wherein the antibody inhibits NLRC5 expression and/or NLRC5 activity.

22. The pharmaceutical composition of claim 20 for the treatment of a disease associated with aberrant expression of MHC class I genes.

23. A method to increase the efficacy and effectiveness of a vaccine by increasing NLRC5 expression and/or NLRC5 activity in a subject in need thereof, the method comprising:
administering to the subject a compound that increases NLRC5 expression and/or NLRC5 activity in an amount effective to increase MHC class I gene expression and increase the efficacy and effectiveness of the vaccine in the subject.

24. The method of claim 23, wherein the compound is selected from the group consisting of siRNA, a synthetic organic chemical, a peptide, a natural fermentation product, and a substance extracted from a microorganism, plant, animal or cell culture medium.


SEQUENCE LISTING (58 sequences)

SEQ ID NO: 1 - protein, 217 aa, Mus musculus
MAARQHSPLLMDAESIRLNNENLWAWLVRLLSKNPEWLSAKLRSFLPTMDLDCSYEPSNEVIHRQLNRLFAQGMATWKSFINDLCFELDVPLDMEIPLVSIWGPRDEFSKQLGAGEECPGPQLYHGAKRPFQSYGSSPRRKNSKKQQLELAKKYLKLLKTSAQQWHGGVCPGAWLTHSPQTYIPPVLQWSRATAPLDAQEGATLGDPEAADNIDVSI

SEQ ID NO: 2 - protein, 206 aa, Homo sapiens
MDPVGLQLGNKNLWSCLVRLLTKDPEWLNAKMKFFLPNTDLDSRNETLDEQRVILQLNKLHVQGSDTWQSFIHCVCMQLEVPLDLEVLLLSTFGYDDGFTSQLGAEGKQPESQLHHGLKRPHQSCGSSPRRKQCKKQQLELAKKYLQLLRTSAQQRYRSQIPGSGQPAFHQVYVPPILRRATASLDTPEGAIMGDVKVEDGADVSI

SEQ ID NO: 3 - protein, 1915 aa, Mus musculus
MDAESIRLNNENLWAWLVRLLSKNPEWLSAKLRSFLPTMDLDCSYEPSNPEVIHRQLNRLFAQGMATWKSFINDLCFELDVPLDMEIPLVSIWGPRDEFSKQLGAGEESCPGPQLYHGAKRPFQSYGSSPRRKNSKKQQLELAKKYLKLLKTSAQQWHGGVCPGAWLTPHSPQTYIPPVLQWSRATAPLDAQEGATLGDPEAADNIDVSIQDLFSFKAHKGPRVTVLLGKAGMGKTTLAYRLRWRWAQGQLDRFQALFLFEFRQLNMITQLPTLPQLLFDLYLMPESEPDAVFQYLKENAQEVLLIFDGLDEALHADSVGTDNAGSALTLFSELCHGNLLPGCWVMTTSRPGKLPSCVPTEAATVHMWGFDGLRVEKYVTCFFSDLLSQELALKEMRTNARLRGMCAIPALCTVTCFCLRRLLPGSSPGQSAALLPTITQLYLQMVETFSPSETLLDTSILGFGKVALRGLDTGKVVFSVEDISPQLMSFGAVHSLLTSFCIHTRPGHEEIGYAFVHLSLQEFFAALYLMASHTVDKDTLVEYVTLNSHWVLRTKGRLGLSDHLPAFLAGLASHTCHMFLCQLAQQDRAWVGSRQAAVIQVLRKLASRKLTGPKMIELYHCVAETQDLELARFTAQSLPSRLSFHNFPLTHADLAALANILEHRDDPIHLDFDGCPLEPHCPEALVGCGQVENLSFKSRKCGDAFAEALCRSLPTMGSLKTLGLTGSRITAQGISHLIQTLPLCSQLEEVSLHDNQLKDPEVLSLVELLPSLPKLQKLDLSRNSFSRSILLSLVKVAITCPTVRKLQVRELDLIFYLSPVTETATQQSGASDVQGKDSLKEGQSRSLQLRLQKCQLRIRDAEALVELFQKSPQLEEVNLSGNHLEDDGCRLVAEAASQLHIAQKLDLSDNGLSQTGVTYVLKAMSTCGTLEDLHISLLNNTVVLTFAQEPREQEGSCKGRAPLISFVSPVTSELSQRSRRIRLTHCGFLAKHTETLCEALRASCQTHNLDHLDLSDNSLGGKGVILLTELLPGLGPLKSLNLSRNGLSMDAVFSLVQCLSSLQWVFHLDVSLESDCIFLRGAGTSRDALEPKFQTGVQVLELSQRYTSRSFCLQECQLEPTSLTFLCATLEKSPGPLEVQLSCKSLSDDSLKILLQCLPQLPQLSLLQLRHTVLSSRSPFLLADIFNLCPRVRKVTLRSLCHAVLHFDSNEEQEGVCCGFPGCSLSQEHMETLCCALSKCNALSQLDLTDNLLGDIGLRCLLECLPQLPISGWLDLSHNNISQEGILYLLETLPSYPNIQEVSVSLSSEQIFRMCFSKKEGAGTSLRLCECSFSPEQVSKLASSLSQAQQLTELWLTKCHLDLPQLTMLLNLVNRPTGLLGLRLEEPWVDSVSLPALMEVCAQASGCLTELSISEIQRKLWLQLEFPHQEGNSDSMALRLAHCDLETEHSHLMIQLVETYARLQQLSLSQVSFNDNDGTSSKLLQNILLSSCELKSFRLTFSQVSTKSLTHLAFGLGHCHHLEELDFSNNSLREEDTELLMGALQGTCRLKKLHLSFLPLGASSLALLIQGLSRMTLLQDLCLSHNQIGDVGTQCLAAILPKLPELRKFDLSHNQIGDVGTQCLAAILPKLPELRKFNLSHNQIGHVGTQCLAAILPKLPELRKFDLSRNQIGDVGTQCLAAILPKLPELRKFDLSGNRIGPAGGVQLVKSLTHFEHLEEIKLGNNALGEPTALELAQRLPPQLRVLCLPSSHLGPEGALGLAQALEQCPHIEEVSLAENNLAGGVPRFSKRLPLLRQIDLEFCKIEDQAARHLAANLTLFPALEKLLLSGNLLGDEVAAELAQVLPQMGQLKKVNLEWNRITARGAQLLAQGLVQGSCVPVIRLWNNPILNDVAQSLQSQEPRLDFSITDQQTL

SEQ ID NO: 4 - protein, 1866 aa, Homo sapiens
MDPVGLQLGNKNLWSCLVRLLTKDPEWLNAKMKFFLPNTDLDSRNETLDPEQRVILQLNKLHVQGSDTWQSFIHCVCMQLEVPLDLEVLLLSTFGYDDGFTSQLGAEGKSQPESQLHHGLKRPHQSCGSSPRRKQCKKQQLELAKKYLQLLRTSAQQRYRSQIPGSGQPHAFHQVYVPPILRRATASLDTPEGAIMGDVKVEDGADVSISDLFNTRVNKGPRVTVLLGKAGMGKTTLAHRLCQKWAEGHLNCFQALFLFEFRQLNLITRFLTPSELLFDLYLSPESDHDTVFQYLEKNADQVLLIFDGLDEALQPMGPDGPGPVLTLFSHLCNGTLLPGCRVMATSRPGKLPACLPAEAAMVHMLGFDGPRVEEYVNHFFSAQPSREGALVELQTNGRLRSLCAVPALCQVACLCLHHLLPDHAPGQSVALLPNMTQLYMQMVLALSPPGHLPTSSLLDLGEVALRGLETGKVIFYAKDIAPPLIAFGATHSLLTSFCVCTGPGHQQTGYAFTHLSLQEFLAALHLMASPKVNKDTLTQYVTLHSRWVQRTKARLGLSDHLPTFLAGLASCTCRPFLSHLAQGNEDCVGAKQAAVVQVLKKLATRKLTGPKVVELCHCVDETQEPELASLTAQSLPYQLPFHNFPLTCTDLATLTNILEHREAPIHLDFDGCPLEPHCPEALVGCGQIENLSFKSRKCGDAFAEALSRSLPTMGRLQMLGLAGSKITARGISHLVKALPLCPQLKEVSFRDNQLSDQVVLNIVEVLPHLPRLRKLDLSSNSICVSTLLCLARVAVTCPTVRMLQAREADLIFLLSPPTETTAELQRAPDLQESDGQRKGAQSRSLTLRLQKCQLQVHDAEALIALLQEGPHLEEVDLSGNQLEDEGCRLMAEAASQLHIARKLDLSDNGLSVAGVHCVLRAVSACWTLAELHISLQHKTVIFMFAQEPEEQKGPQERAAFLDSLMLQMPSELPLSSRRMRLTHCGLQEKHLEQLCKALGGSCHLGHLHLDFSGNALGDEGAARLAQLLPGLGALQSLNLSENGLSLDAVLGLVRCFSTLQWLFRLDISFESQHILLRGDKTSRDMWATGSLPDFPAAAKFLGFRQRCIPRSLCLSECPLEPPSLTRLCATLKDCPGPLELQLSCEFLSDQSLETLLDCLPQLPQLSLLQLSQTGLSPKSPFLLANTLSLCPRVKKVDLRSLHHATLHFRSNEEEEGVCCGRFTGCSLSQEHVESLCWLLSKCKDLSQVDLSANLLGDSGLRCLLECLPQVPISGLLDLSHNSISQESALYLLETLPSCPRVREASVNLGSEQSFRIHFSREDQAGKTLRLSECSFRPEHVSRLATGLSKSLQLTELTLTQCCLGQKQLAILLSLVGRPAGLFSLRVQEPWADRARVLSLLEVCAQASGSVTEISISETQQQLCVQLEFPRQEENPEAVALRLAHCDLGAHHSLLVGQLMETCARLQQLSLSQVNLCEDDDASSLLLQSLLLSLSELKTFRLTSSCVSTEGLAHLASGLGHCHHLEELDLSNNQFDEEGTKALMRALEGKWMLKRLDLSHLLLNSSTLALLTHRLSQMTCLQSLRLNRNSIGDVGCCHLSEALRAATSLEELDLSHNQIGDAGVQHLATILPGLPELRKIDLSGNSISSAGGVQLAESLVLCRRLEELMLGCNALGDPTALGLAQELPQHLRVLHLPFSHLGPGGALSLAQALDGSPHLEEISLAENNLAGGVLRFCMELPLLRQIDLVSCKIDNQTAKLLTSSFTSCPALEVILLSWNLLGDEAAAELAQVLPQMGRLKRVDLEKNQITALGAWLLAEGLAQGSSIQVIRLWNNPIPCDMAQHLKSQEPRLDFAFFDNQPQAPWGT

SEQ ID NO: 5 - DNA, 38 bp, Artificial sequence
  Synthetic oligonucleotide
cacagcatccttagacactccggagggggccattatgg

SEQ ID NO: 6 - DNA, 39 bp, Artificial sequence
  Synthetic oligonucleotide
ccataatggccccctccggagtgtctaaggatgctgtgg

SEQ ID NO: 7 - DNA, 28 bp, Artificial sequence
  Synthetic oligonucleotide
atatagatctgaccccgttggcctccag

SEQ ID NO: 8 - DNA, 31 bp, Artificial sequence
  Synthetic oligonucleotide
atattctagatcaagtaccccaaggggcctg

SEQ ID NO: 9 - DNA, 28 bp, Artificial sequence
  Synthetic oligonucleotide
atatagatctgaccccgttggcctccag

SEQ ID NO: 10 - DNA, 38 bp, Artificial sequence
  Synthetic oligonucleotide
atatgaattcttagcccttgttaaccctggtgttgaag

SEQ ID NO: 11 - DNA, 28 bp, Artificial sequence
  Synthetic oligonucleotide
atatagatctgagttggccaagaagtac

SEQ ID NO: 12 - DNA, 31 bp, Artificial sequence
  Synthetic oligonucleotide
atattctagattaagtaccccaaggggcctg

SEQ ID NO: 13 - DNA, 28 bp, Artificial sequence
  Synthetic oligonucleotide
atatagatctgagttggccaagaagtac

SEQ ID NO: 14 - DNA, 31 bp, Artificial sequence
  Synthetic oligonucleotide
atattctagattagctgagattctctatctg

SEQ ID NO: 15 - DNA, 29 bp, Artificial sequence
  Synthetic oligonucleotide
atatagatcttttaagagcaggaagtgtg

SEQ ID NO: 16 - DNA, 31 bp, Artificial sequence; identical to SEQ ID NO: 12
  Synthetic oligonucleotide

SEQ ID NO: 17 - DNA, 28 bp, Artificial sequence
  Synthetic oligonucleotide
atatagatctgaccccgttggcctccag

SEQ ID NO: 18 - DNA, 31 bp, Artificial sequence; identical to SEQ ID NO: 14
  Synthetic oligonucleotide

SEQ ID NO: 19 - DNA, 46 bp, Artificial sequence
  Synthetic oligonucleotide
gagctgtgggtcctcacccgccgcggcgcagtgcaagaagcagcag

SEQ ID NO: 20 - DNA, 46 bp, Artificial sequence
  Synthetic oligonucleotide
ctgctgcttcttgcactgcgccgcggcgggtgaggacccacagctc

SEQ ID NO: 21 - DNA, 41 bp, Artificial sequence
  Synthetic oligonucleotide
cagctccaccatggcctggcggccccacatcagagctgtgg

SEQ ID NO: 22 - DNA, 41 bp, Artificial sequence
  Synthetic oligonucleotide
ccacagctctgatgtggggccgccaggccatggtggagctg

SEQ ID NO: 23 - DNA, 37 bp, Artificial sequence
  Synthetic oligonucleotide
ggaaggctggcatgggcgcgaccacgctggcccaccg

SEQ ID NO: 24 - DNA, 37 bp, Artificial sequence
  Synthetic oligonucleotide
cggtgggccagcgtggtcgcgcccatgccagccttcc

SEQ ID NO: 25 - DNA, 42 bp, Artificial sequence
  Synthetic oligonucleotide
gatctttgatgggctagatcaggccctccagcctatgggtcc

SEQ ID NO: 26 - DNA, 42 bp, Artificial sequence
  Synthetic oligonucleotide
ggacccataggctggagggcctgatctagcccatcaaagatc

SEQ ID NO: 27 - DNA, 34 bp, Artificial sequence
  Synthetic oligonucleotide
atatggatccatggacgctgagagcatccgactg

SEQ ID NO: 28 - DNA, 33 bp, Artificial sequence
  Synthetic oligonucleotide
atatatctagatcaaagagtctgctggtcagtg

SEQ ID NO: 29 - DNA, 21 bp, Artificial sequence
  Synthetic oligonucleotide
ctggccagtctcaccgcacaa

SEQ ID NO: 30 - DNA, 22 bp, Artificial sequence
  Synthetic oligonucleotide
ccaggggacagccatcaaaatc

SEQ ID NO: 31 - DNA, 23 bp, Artificial sequence
  Synthetic oligonucleotide
aaaaggagggagttacactcagg

SEQ ID NO: 32 - DNA, 21 bp, Artificial sequence
  Synthetic oligonucleotide
gctgtgagggacacatcagag

SEQ ID NO: 33 - DNA, 17 bp, Artificial sequence
  Synthetic oligonucleotide
ctaccctgcggagatca

SEQ ID NO: 34 - DNA, 18 bp, Artificial sequence
  Synthetic oligonucleotide
acagccaggccagcaaca

SEQ ID NO: 35 - DNA, 24 bp, Artificial sequence
  Synthetic oligonucleotide
cacacctctcctttgtgacttcaa

SEQ ID NO: 36 - DNA, 24 bp, Artificial sequence
  Synthetic oligonucleotide
ccacctcctcacattatgctaaca

SEQ ID NO: 37 - DNA, 21 bp, Artificial sequence
  Synthetic oligonucleotide
agggctggctggctgctttga

SEQ ID NO: 38 - DNA, 22 bp, Artificial sequence
  Synthetic oligonucleotide
acgtggcccatggtgttgttat

SEQ ID NO: 39 - DNA, 22 bp, Artificial sequence
  Synthetic oligonucleotide
cgttgtgatgggttctgattcc

SEQ ID NO: 40 - DNA, 23 bp, Artificial sequence
  Synthetic oligonucleotide
gacagcttgtcaaacactcggtt

SEQ ID NO: 41 - DNA, 25 bp, Artificial sequence
  Synthetic oligonucleotide
tgctgtctccatgtttgatgtatct

SEQ ID NO: 42 - DNA, 22 bp, Artificial sequence
  Synthetic oligonucleotide
tctctgctccccacctctaagt

SEQ ID NO: 43 - DNA, 21 bp, Artificial sequence
  Synthetic oligonucleotide
gccaacctggaaatcatgaca

SEQ ID NO: 44 - DNA, 19 bp, Artificial sequence
  Synthetic oligonucleotide
agggctgttcgtgagcaca

SEQ ID NO: 45 - DNA, 19 bp, Artificial sequence
  Synthetic oligonucleotide
ggctggaatttggcagcac

SEQ ID NO: 46 - DNA, 21 bp, Artificial sequence
  Synthetic oligonucleotide
gcccaacacaaggatgtctct

SEQ ID NO: 47 - DNA, 21 bp, Artificial sequence
  Synthetic oligonucleotide
ccatcctttggtacaacatgc

SEQ ID NO: 48 - DNA, 19 bp, Artificial sequence
  Synthetic oligonucleotide
tgcacatggtggagtcagg

SEQ ID NO: 49 - DNA, 18 bp, Artificial sequence
  Synthetic oligonucleotide
gaaggtgaaggtcggagt

SEQ ID NO: 50 - DNA, 20 bp, Artificial sequence
  Synthetic oligonucleotide
gaagatggtgatgggatttc

SEQ ID NO: 51 - DNA, 20 bp, Artificial sequence
  Synthetic oligonucleotide
tccgcagtttcttttctccc

SEQ ID NO: 52 - DNA, 21 bp, Artificial sequence
  Synthetic oligonucleotide
ggagaatctgagtcccggtgg

SEQ ID NO: 53 - DNA, 22 bp, Artificial sequence
  Synthetic oligonucleotide
tctcagggtctcaggctccgag

SEQ ID NO: 54 - DNA, 22 bp, Artificial sequence
  Synthetic oligonucleotide
tgcgtggggactttagaactgg

SEQ ID NO: 55 - DNA, 27 bp, Artificial sequence
  Synthetic oligonucleotide
atttttctgattggccaaagagtaatt

SEQ ID NO: 56 - DNA, 26 bp, Artificial sequence
  Synthetic oligonucleotide
aaaagaaaagagaatgtggggtgtaa

SEQ ID NO: 57 - DNA, 20 bp, Artificial sequence
  Synthetic oligonucleotide
tactagcggttttacgggcg

SEQ ID NO: 58 - DNA, 24 bp, Artificial sequence
  Synthetic oligonucleotide
tcgaacaggaggagcagagagcga